SEQUENCE LISTING (2 sequences)

SEQ ID NO: 1 - DNA, 20 bp, Artificial Sequence
  primer MGMT F
  primer_bind (1)..(20): y stands for c or t
tygygttttggatatgttgg

SEQ ID NO: 2 - DNA, 20 bp, Artificial Sequence
  primer MGMT R
  primer_bind (1)..(20): r stamds for c or t
craaaaaaaactccrcactc

(19) United States
(12) Patent Application Publication
(10) Pub. No.: US 2022/0213555 A1
(43) Pub. Date: Jul. 7, 2022

(54) NEXT GENERATION SEQUENCING-BASED DETECTION PANEL FOR GLIOMA, DETECTION KIT, DETECTION METHOD AND APPLICATION THEREOF

(71) Applicant: GENECAST (WUXI) PRECISION MEDICAL DIGNOSTIC LABORATORY, Wuxi, Jiangsu (CN)

(72) Inventors: Yuanyuan HONG, WuXi, Jiangsu (CN); Jianing YU, WuXi, Jiangsu (CN); Xianchao GUO, WuXi, Jiangsu (CN); Huiting YAN, WuXi, Jiangsu (CN); Xiaofeng SONG, WuXi, Jiangsu (CN); Caiqin LI, WuXi, Jiangsu (CN); Minjun CHEN, WuXi, Jiangsu (CN); Xin LI, WuXi, Jiangsu (CN); Weizhi CHEN, WuXi, Jiangsu (CN); Ji HE, WuXi, Jiangsu (CN)

(21) Appl. No.: 17/609,418

(22) PCT Filed: Sep. 19, 2019
(86) PCT No.: PCT/CN2019/106606

§ 371 Date: Nov. 8, 2021

(30) Foreign Application Priority Data
May 6, 2019 (CN) 201910372726.0
May 6, 2019 (CN) 201910373154.8
May 6, 2019 (CN) 201910373158.6

(51) Int. Cl. C12Q 1/6886; C12Q 1/6858; C12Q 1/6869

(52) U.S. Cl. CPC C12Q 1/6886; C12Q 1/6869; C12Q 1/6858

(57) ABSTRACT

Disclosed are a next generation sequencing-based detection panel for glioma, a detection kit, a detection method and an application thereof. The detection panel contains glioma-related genes and loci, wherein the glioma-related genes and loci contain SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPMID, TP53, BCOR, DDX3X, IDH1, MYC, PTCH1 and the like.

### CROSS-REFERENCES TO RELATED APPLICATION

The present application is a National Stage of International Patent Application No: PCT/CN2019/106606 filed on Sep. 19, 2019, which claims the benefit of the priority of the Chinese patent application with the application No. 201910373154.8, filed to the China National Intellectual Property Administration on May 6, 2019, the application No. 201910373158.6, titled filed to the China National Intellectual Property Administration on May 6, 2019, the application No. 201910372726.0, filed to the China National Intellectual Property Administration on May 6, 2019, the entire contents of which are incorporated in this application by reference.

### SEQUENCE LISTING

The present application contains a Sequence Listing which has been submitted electronically in ASCII format and is hereby incorporated by reference in its entirety. Said ASCII copy is named_Sequence_Listing.txt and is 4.0 kilobytes in size, and contains 2 sequences from SEQ ID NO:1 to SEQ ID NO:2, which are identical to the sequence listing filed in the corresponding international application No. PCT/CN2019/106606 filed on Sep. 19, 2019.

### TECHNICAL FIELD

The present disclosure relates to the technical field of biomedicine, in particular to a next generation sequencing-based detection panel for glioma, a detection kit, a detection method and an application thereof.

### BACKGROUND

The high-throughput sequencing technology is a revolutionary change to the conventional one generation sequencing, which includes preparing a sequencing library from DNA connecting adapters, conducting extension reaction on tens of thousands of cloning in the library, detecting corresponding signals and finally obtaining sequence information. Hundreds of thousands of or millions of DNA molecules can be sequenced at a time, such that the high-throughput sequencing technology is referred to as the next generation sequencing (NGS). Meanwhile, it is possible to analyze transcriptomes and genomes of one species by high-throughput sequencing in an overall view manner meticulously, and therefore, the high-throughput sequencing technology is also referred to as the deep sequencing.

The NGS detection method is high in throughput, so it can detect a lot of genes, meet the demands on clinical detection, and can not only detect known mutation sites, but also unknown mutation sites. In addition, the NGS detection method further can detect various mutation types, and various clinical samples, such as full blood, tissue, FFPE sample and cfDNA and the other sample types.

At present, major sequencing technical platforms are mainly divided into:

(1) Solexa sequencing technology: a mainstream illumina sequencing platform; (2) 454 sequencing technology: long-read, but low accuracy and high cost, and pyrosequencing technology, low market share; and

(3) Solid sequencing technology: double-color encoding technology.

The targeted sequence capturing sequencing technology (Targeted Resequencing) is a detection method, which includes the steps of designing a specific probe for an interested genome region, hybridizing the specific probe with genomic DNA, enriching DNA fragments in a targeted genome region and conducting sequencing by means of the high throughput sequencing technology.

Glioma is the most common intracranial primary malignant tumor. In adults, glioma accounts for about 30-40% of all brain tumors. In primary malignant central nervous system tumors, glioblastoma (GBM) has the highest morbidity, accounting for 46.1% which is about 3.20/100000, the median onset age is 65 years old, the median overall survival is 14.6 months and the therapeutic effect is not ideal. Glioma is clinically characterized in high morbidity, high postoperative recurrence and low cure rate.

According to level of similarity of tumor cells and normal brain gliocytes in morphology (not necessarily a true cell origin), Glioma can be divided into astrocytoma-astrocytes, ooligodendrocytoma-oligodendrocytes, ependymoma-ependymocytes and mixed glioma—for example, ooligodendrocytom and astrocytoma, including gliocytes of hybrid types.

According to a grading system formulated by WHO, glioma can be divided into grade 1 (with lowest grade malignancy and best prognosis) to grade 4 (with highest grade malignancy and poorest prognosis) according to grade malignancy of tumor cells. So called anaplastic glioma in conventional cytopathology corresponds to grade 3 of WHO, and glioblastoma corresponds to grade 4 of WHO. According to the grading system, glioma can be further classified as follows according to the grade malignancy of the tumor cells in pathology:

1) low-grade glioma (WHO Grade I-II) is a well differentiated glioma: although the tumor is not a benign tumor biologically, a patient is relatively better in prognosis;

2) high-grade glioma (WHO Grade III-IV) is a poorly differentiated glioma: the tumor is a malignant tumor, and a patient is relatively poor in prognosis.

In the Central Nervous System Tumor Classification, WHO, edition 2016, molecular features are added based on histology for the first time, and comprehensive diagnosis is adopted. The classification integrates histopatholoic and genotype parameters, such that accuracy in parting, diagnosis, prognosis and treatment decision of glioma is improved.

In conventional detection of glioma-related genes, it is necessary to combine a plurality of experimental platforms and instruments and equipment. For example, a conventional IDH mutation detection method is immunohistochemical (IHC), a 1p19q detection method is a fluorescence in situ hybridization (FISH) and STR identification, and a MGMT promoter methylation detection method is a methylated specific PCR (MSP) and pyrosequencing. Correspondingly, it is necessary to be equipped with a one-generation sequencer, a pyrosequencer, a fluorescence microscope, a qPCR meter and the like, and meanwhile, it is also necessary to purchase corresponding reagents. Many instruments and kits are equipped for finishing whole set of detection, and meanwhile, it is needed to operate each detection method by corresponding professionals, so that the overall input cost is very high.

Fluorescence in situ hybridization is a golden standard method for detecting co-deletion of 1p/19q in a glioma sample in clinical pathology at present. It is difficult to prepare and band a solid tumor chromosome and it is operated by professionals rich in experience. Moreover, the probes are limited in quantity, small in throughput and long in time. Deletion on a small portion of fixed positions on 1p and 19q can be detected and a condition on an entire chromosome arm cannot be detected within a larger scope, like NGS. Besides, there is a large deviation on result judgment according to different labs and detection mechanisms.

One-generation electrocapillary phoresis is a mature molecular biological technique at present. It is needed to judge whether deletion is available or not according to a condition whether amplified fragments are available or not by aligning with DNA of blood cells or DNA of normal cells of a testee. As judgment of deletion by detecting small portions of fixed STR regions on 1p and 19q is not larger than NGS judgment scope, and if homozygous conditions are available in the STR regions, the homozygotic conditions cannot be included in a judgment result, such that the result accuracy is reduced. It is tedious to operate, and most results are judged subjectively by experimenters, so that the results cannot be judged conveniently and accurately.

MGMT is a DNA repair protein prevailing in cells and can remove O⁶ guanine compounds from DNA, so that the damaged guanines are recovered, and therefore, the chromosome is prevented from being damaged by an alkylating agent. In the process, MGMT not only serves as transmethylase, but also serves as a methyl receptor protein, and thereby, a transfer reaction is completed independently.

The MGMT gene promoter methylation state is of certain relevance to sensitivity of an alkylating agent medicine. The alkylating agents: temozolomide (TMZ), nimustine (ACNU) and dichloroethyl nitrourea (BCNU) and the like are widely applied to treating human tumors as chemotherapeutics. An important action site of each of those alkylating agents is O⁶ guanine and MGMT can remove alkyl compounds on the O⁶ guanine quickly, so that the curative effect of the alkylating agent in killing tumors is reduced, and therefore, tumors are drug-resistant.

Thus, detection of MGMT gene promoter methylation state is beneficial to predict the sensitivity of tumors to the chemotherapeutics: alkylating agents, so that it is favorable to guide and formulate a chemotherapeutic scheme to prevent drug resistance.

However, at present, a common method for detecting MGMT promotor methylation is hydrosulphite sequencing PCR (BSP), methylation specific PCR (MSP), fluorescence quantitative method and methylation sensitivity high resolution melting curve analysis (MS-HRM).

The hydrosulphite sequencing PCR (BSP) method detects a methylated state primarily by means of PCR combining with a sanger sequencing technology. However, as the method is tedious to operate and long in detection period, the method is not suitable for large batch detection. Meanwhile, selection of cloning quantity may lead to a false positive result, and therefore, BSP is only considered as a semiquantitative method.

The methylation specific PCR (MSP) method judges whether methylation exists or not on the sample by PCR amplification. The method is practical and is widely applied. But the method cannot be used for quantitative detection and has a higher false positive risk.

The fluorescence quantitative method is a technology developed based on MSP, and the key point is that a TaqMan probe is added in a detection process, so that higher sensitivity and accuracy are guaranteed. However, if more methylation loci are detected, the fluorescence quantitative method only can perform integrated analysis. Meanwhile, the probe is high in cost, so that the method is not suitable for detecting a large amount of samples with more loci.

The MS-HRM converts a variation between single base sequences into a variation of the melting curve to judge whether methylation exists or not. However, the method is quite high in demand on instrument and needs a fluorescent quantitative PCR instrument with an HRM module.

The method only can be used for analyzing the entire methylation state of the fragment but cannot be used for specifying the methylation state of each CpG locus. Therefore, it is still necessary to provide an efficient and accurate detection scheme for detecting the methylation state of the MGMT gene.

### SUMMARY

The present disclosure aims to provide a next generation sequencing-based detection panel for glioma, a detection kit, a detection method and an application thereof so as to provide a detection method or product for glioma with a low cost.

In order to achieve the foregoing purpose, according to one aspect of the present disclosure, a next generation sequencing-based detection panel for glioma is provided. The detection panel includes glioma-related genes and loci. The glioma-related genes and loci include SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPM1D, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR vIII, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1.

Further, the glioma-related genes and loci further include STR locus on chromosome 1 and STR locus on chromosome 19.

According to another aspect of the present disclosure, a next generation sequencing-based detection kit for glioma is provided. The detection kit includes a detection probe and/or a detection primer directed at glioma-related genes and loci, the glioma-related genes and loci including SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPM1D, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR vIII, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1.

Further, the glioma-related genes and loci further include STR locus on chromosome 1 and STR locus on chromosome 19.

Further, the detection kit is used for detecting a plurality of mutation types, the plurality of mutation types including point mutation, fusion mutation, copy number variation, deletion mutation and insertion mutation.

Further, the detection kit further includes primers for detecting the MGMT promoter methylation, wherein the primers for detecting the MGMT promoter methylation have sequences shown by SEQ ID NO: 1 and SEQ ID NO: 2.

Further, the detection kit further includes one or more of groups being comprised of DNA library construction reagents, gene trapping reagents, bisulfite conversion reagents and gene amplification reagents.

Further, the detection kit further includes a glioma panel verification sample, the glioma panel verification sample including IDH1, IDH2, TERT, ABL1, ALK, BRAF, EGFR, FGFR2, FLT3, GNA11, GNA11, GNAQ, JAK2, KIT, KRAS, MEK1, MET, NOTCH, NRAS, PDGFRA, PIK3CA and NTRK gene standard substances.

Further, the detection kit further includes a next generation sequencing-based system for detecting 1p/19q co-deletion of glioma, the next generation sequencing-based system for detecting 1p/19q co-deletion of glioma including an SNP locus selection device, an SNP detection device without a control sample and/or an SNP detection device with the control sample, wherein the SNP locus selection device is configured to select the SNP loci on chromosome 1 and chromosome 19 according public databases to obtain a first group of SNP loci; the SNP detection device without the control sample includes: a first sequencing module configured for sequencing a to-be-tested sample and a group of negative samples; a first SNP detection module configured for detecting all SNP loci on chromosome 1 and chromosome 19 in the group of negative samples; a first gSNP locus selection module configured for selecting gSNP loci of the group of negative samples in the first group of SNP loci; a second SNP detection module configured for detecting all SNP loci on chromosome 1 and chromosome 19 in the to-be-tested sample; a first calculation and statistics module configured for performing calculation and statistics on BAF of mutated gSNP loci on the gSNP loci determined in the first gSNP loci selection module in the to-be-tested sample and marking the LOH status ratio (Ri) of the ith gSNP as |BAF−0.5| of the ith gSNP; and a first judging module configured for correcting R on 1p and 19q of the to-be-tested sample according to R of the gSNP loci on 1q and 19p of the to-be-tested sample and determining a threshold value, judging the LOH status of each gSNP locus according to the threshold value and then judging co-deletion according to LOH statuses of all gSNP loci;

the SNP detection device with the control sample includes: a second sequencing module configured for sequencing the to-be-tested sample and a control sample; a third SNP detection module configured for detecting all SNP loci on chromosome 1 and chromosome 19 in the control sample; a second gSNP locus selection module configured for selecting gSNP loci of the control sample in the first group of SNP loci; a fourth SNP detection module configured for detecting all SNP loci on chromosome 1 and chromosome 19 in the to-be-tested sample; a second calculation and statistics module configured for performing statistics on numbers of reads of reference sequence genotype and alternative sequence genotype of the control sample on the gSNP loci and marking as N₁ and N₂ respectively, performing statistics on numbers of reads of reference sequence genotype and alternative sequence genotype of the to-be-detected sample on the gSNP loci and marking as T₁ and T₂ respectively, calculating an LOH status ratio of each gSNP, wherein the LOH status (Rⁱ) of the ith gSNP is defined as follows:

$${R}^{i} = \vert \frac{\frac{{N}_{2}^{i}}{{N}_{1}^{i}}}{\frac{{N}_{2}^{i}}{{N}_{1}^{i}} + \frac{{T}_{2}^{i}}{{T}_{1}^{i}}} - 0.5 \vert ;$$

and a second judging module configured for correcting R on 1p and 19q of the to-be-tested sample according to R of the gSNP loci on 1q and 19p of the to-be-tested sample and determining a threshold value, judging the LOH status of each gSNP locus according to the threshold value and then judging co-deletion according to LOH statuses of all gSNP loci.

Further, the first judging module includes: a first statistics sub-module configured for performing statistics on mean values and variances of R in all gSNP loci in 1q and 19p respectively and calculating a Z value of each R on chromosome 1 and chromosome 19 respectively based on 1q and 19p; a first threshold value calculation sub-module configured for calculating the Z values of the group of negative samples corrected by 1q and 19p and taking the mth percentile as a threshold value, preferably, m is greater than 95, and more preferably, m is equal to 99; a first judging sub-module configured for comparing the Z value of each gSNP locus on 1p and 19q with the corresponding threshold value to judge the LOH status of the locus, judging the LOH status of the locus abnormal if the Z value exceeds the threshold value, otherwise judging the LOH status of the locus normal; and a second judging sub-module configured for judging whether LOH occurs on 1p and 19q or not, performing statistics on numbers of abnormal and normal statuses on 1p and 19q respectively, judging that the LOH of the sample occurs on 1p and 19q when abnormal statuses/(abnormal statuses+normal statuses) is greater than t₁, and judging co-deletion of 1p and 19q of the sample occurs when only LOH occurs on 1p and 19q simultaneously, preferably, t₁ is greater than 0.6, and more preferably, t₁ is equal to 0.8.

Further, the first gSNP locus selection module selects the gSNP loci of the group of negative samples in the first group of SNP loci according to a coverage, BAF and a fluctuation size of BAF in the group of negative samples, preferably, selection criteria of the gSNP loci are as follows: a coverage is greater than 100, a BAF range is 0.1-0.9 and max-min of BAF among samples in the group of negative samples is smaller than 0.2; and preferably, the number of samples in the group of negative samples is greater than or equal to 30.

Further, the second judging module includes: a second statistics sub-module configured for performing statistics on mean values and variances of R in all gSNP loci in 1q and 19p respectively and calculating a Z value of each R on chromosome 1 and chromosome 19 respectively based on 1q and 19p; a second threshold value calculation sub-module configured for taking the mean values of Z values on 1q and 19p plus 2-6 times of variances respectively as threshold values of 1p and 19q; a third judging sub-module configured for comparing the Z value of each gSNP locus on 1p and 19q with the corresponding threshold value to judge the LOH status of the locus, judging the LOH status of the locus abnormal if the Z value exceeds the threshold value, otherwise judging the LOH status of the locus is normal; and a fourth judging sub-module configured for judging whether the LOH occurs on 1p and 19q or not, performing statistics on numbers of abnormal and normal statuses on 1p and 19q respectively, judging that the LOH of the sample occurs on 1p/19q when abnormal statuses/(abnormal statuses-+normal statuses) is greater than t₂, and judging co-deletion of 1p and 19q of the sample occurs when only LOH occurs on 1p and 19q simultaneously, preferably, t₂ is greater than 0.6, and more preferably, t₂ is equal to 0.9.

Further, the second gSNP locus selection module selects the gSNP loci of the control sample in the first group of SNP loci according to a coverage and BAF, preferably, selection criteria of the gSNP loci are as follows: the coverage is greater than 100, and the BAF range is 0.3-0.7.

Further, the public databases include SNP138, 1000 Genomes Project and Chinese Millionome Database, preferably, the SNP locus selection device selecting the loci SNP loci according to their allele frequency between 0.45-0.55 in the population; and preferably, an SNP locus being selected at every 200 kb.

Further, the system includes a first verification device, the first verification device being configured to detect co-deletion of 1P and 19q based on STR and the first verification device including: an STR acquisition module configured for extracting known STR from existing data; a control sample STR statistics module configured for extracting reads covering the known STR from an alignment result file of the control sample, performing statistics on the number of repeat unit of the known STR on each read, extracting the two repeat units with the most numbers for each STR, marking as N₃ and N₄, wherein if

$$\frac{{N}_{3}}{{N}_{4}}$$

is greater than n, it is considered that the STR is a homozygotic type and is no longer configured for result judgment; and preferably, n is greater than 5, and more preferably, n is equal to 10; a to-be-tested sample STR statistics module configured extracting reads covering the known STR from an alignment result file of the to-be-tested sample, performing statistics the number of reads on the two repeat units determined in the control sample STR statistics module marking as T₃ and T₄ and calculating an LOH status of each STR, wherein the LOH status (Rⁱ) of the ith STR is defined as follows:

$${R}^{i} = \frac{{T}_{4}^{i} / {T}_{3}^{i}}{{N}_{4}^{i} / {N}_{3}^{i}} ,$$

and a third judging module configured for correcting R on 1p and 19q of the to-be-tested sample and determining a threshold value, judging the LOH status of each STR locus according to the threshold value and then judging co-deletion according to LOH statuses of all STR loci; preferably, the reads covering the known STR are reads covering from the upstream 20 bp to the downstream 20 bp of the known STR.

Further, the third judging module includes: a fifth judging sub-module configured for judging the LOH status of each STR and judging that the LOH status of the locus is abnormal when R is smaller than T, otherwise, judging that the LOH status of the locus is normal, preferably, T is equal to 0.5; if R is greater than 1, converting to 1/R; a sixth judging sub-module configured for judging whether LOH occurs on 1p and 19q or not, performing statistics on numbers of abnormal and normal statuses on 1p and 19q respectively, judging that the LOH of the sample occurs on 1p/19q when abnormal statuses/(abnormal statuses+normal statuses) is greater than t₃, and judging co-deletion of 1p and 19q of the sample when only LOH occurs on 1p and 19q simultaneously, preferably, t₃ is greater than 0.6, and more preferably, t₃ is equal to 0.8.

Further, the system includes a second verification device, the second verification device being configured to detect co-deletion of 1p and 19q based on CNV.

Further, the detection kit further includes a processing device for sequencing data of MGMT gene promoter methylation, wherein the processing device for sequencing data of MGMT gene promoter methylation includes: an acquisition module configured for acquiring methylated sequencing data originated from an MGMT gene promoter, wherein the methylated sequencing data is a double-end sequencing sequence; an alignment module configured for aligning the methylated sequencing data with a human reference genomic sequence to obtain an alignment result, the alignment result including a first end first matching region, a first end second matching region, a second end first matching region and a second end matching region, wherein the first end second matching region and the second end second matching region are overlapped; a removal module configured for removing the first end second matching region or the second end second matching region in the alignment result to obtain to-be-analyzed data; and a methylation recognition module configured for methylated locus recognition in the to-be-analyzed data to obtain a methylated result of the MGMT gene promoter.

Further, the processing device further includes: a first pre-treatment module configured for pre-treatment of converting the human reference genomic sequence from C to T; and a second pre-treatment module configured for pre-treatment of converting the double-end sequencing sequence from C to T.

Further, the processing device further includes a correction module configured for correcting the to-be-analyzed data, the correction module being configured to correct the to-be-analyzed data by means of the human reference genomic sequence, position information of the human reference genomic sequence and high frequency SNP loci in a population.

Further, the methylated recognition module includes: a primary identification module configured for identifying the methylated loci in the to-be-analyzed data primarily to obtain primarily identified loci; and a confidence selection module configured for confidence selection of the primarily identified loci to obtain a methylated result of the MGMT gene promoter, preferably, criteria for the confidence selection are as follows: a coverage is smaller than 3000000, a possibility ratio standard between optimum and sub-optimum genotypes is greater than or equal to 20 and a comparison mass is greater than 5.

According to yet another aspect of the present disclosure, use of a next generation sequencing-based detection panel for glioma or the next generation sequencing-based detection kit for glioma in selecting drugs for treating or relieving glioma is provided.

Further, the drug for treating or relieving glioma includes a targeted drug, a chemotherapeutic drug or an immune drug.

According to yet another aspect of the present disclosure, a method for detecting glioma is provided. A method detecting for glioma includes detecting the glioma-related genes and loci by using a detection probe and/or a detection primer, the glioma-related genes and loci including SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPM1D, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR vIII, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1.

Further, the glioma-related genes and loci further include STR locus on chromosome 1 and STR locus on chromosome 19.

Further, the detection method further includes detecting a plurality of mutation types, the plurality of mutation types including point mutation, fusion mutation, copy number variation, deletion mutation and insertion mutation.

Further, the detection method further includes detecting the MGMT promoter methylation, wherein the primers for detecting the MGMT promoter methylation have sequences shown by SEQ ID NO: 1 and SEQ ID NO: 2.

Further, the detection method further includes detecting 1p/19q co-deletion of glioma based on next generation sequencing, the detecting 1p/19q co-deletion of glioma based on next generation sequencing including selecting the SNP loci and performing SNP detection without the control sample and/or SNP detection with the control sample, wherein selecting the SNP loci includes selecting the SNP loci on chromosome 1 and chromosome 19 of human according to the public databases to obtain the first group of SNP loci; and the SNP detection without the control sample includes: S11, sequencing a to-be-tested sample and a group of negative samples; S12, detecting all SNP loci on chromosome 1 and chromosome 19 in the group of negative samples; S13, selecting gSNP loci of the group of negative samples in the first group of SNP loci; S14, detecting all SNP loci on chromosome 1 and chromosome 19 in the to-be-tested sample; S15, performing calculation and statistics on BAF of mutated gSNP loci on the gSNP loci determined in the S13 in the to-be-tested sample and marking the LOH status ratio (Rⁱ) of the ith gSNP as |BAF−0.5| of the ith gSNP; and S16, correcting R on 1p and 19q of the to-be-tested sample according to R of the gSNP loci on 1q and 19p of the to-be-tested sample and determining a threshold value, judging the LOH status of each gSNP locus according to the threshold value and then judging co-deletion according to LOH statuses of all gSNP loci; the SNP detection device with the control sample includes: S21, sequencing the to-be-tested sample and the control sample; S22, detecting all SNP loci on chromosome 1 and chromosome 19 in the control sample; S23, selecting gSNP loci of the control sample in the first group of SNP loci; S24, detecting all SNP loci on chromosome 1 and chromosome 19 in the to-be-tested sample; S25, performing statistics on numbers of reads of reference sequence genotype and alternative sequence genotype of the control sample on the gSNP loci and marking as N₁ and N₂ respectively, performing statistics on numbers of reads of reference sequence genotype and alternative sequence genotype of the to-be-detected sample on the gSNP loci and marking as T₁ and T₂ respectively, calculating an LOH status ratio of each gSNP, wherein an LOH status (Rⁱ) of the ith gSNP is defined as follows:

$${R}^{i} = \vert \frac{\frac{{N}_{2}^{i}}{{N}_{1}^{i}}}{\frac{{N}_{2}^{i}}{{N}_{1}^{i}} + \frac{{T}_{2}^{i}}{{T}_{1}^{i}}} - 0.5 \vert ;$$

and

S26, correcting R on 1p and 19q of the to-be-tested sample according to R of the gSNP loci on 1q and 19p of the to-be-tested sample and determining a threshold value, judging the LOH status of each gSNP locus according to the threshold value and then judging co-deletion according to LOH statuses of all gSNP loci.

Further, the S16 includes: S161, performing statistics on mean values and variances of R in all gSNP loci in 1q and 19p respectively and calculating a Z value of each R on chromosome 1 and chromosome 19 respectively based on 1q and 19p; S162, calculating the Z values of the group of negative samples corrected by 1q and 19p and taking the mth percentile as a threshold value, preferably, m is greater than 95, and more preferably, m is equal to 99; S163, aligning the Z value of each gSNP locus on 1p and 19q with the corresponding threshold value to judge the LOH status of the locus, judging the LOH status of the locus abnormal if the Z value exceeds the threshold value, otherwise judging the LOH status of the locus normal; and S164, judging whether LOH occurs on 1p and 19q or not, performing statistics on numbers of abnormal and normal statuses on 1p and 19q respectively, judging that the LOH of the sample occurs on 1p and 19q when abnormal statuses/(abnormal statuses+normal statuses) is greater than t1, and judging co-deletion of 1p and 19q of the sample when only LOH occurs on 1p and 19q simultaneously, preferably, t₁ is greater than 0.6, and more preferably, t₁ is equal to 0.8.

Further, the S13 includes selecting the gSNP loci of the group of negative samples in the first group of SNP loci according to a coverage, BAF and a fluctuation size of BAF in the group of negative samples, preferably, selection criteria of the gSNP loci are as follows: the coverage is greater than 100, the BAF range is 0.1-0.9 and max-min of BAF among samples in the group of negative samples is smaller than 0.2; and preferably, the number of samples in the group of negative samples is greater than or equal to 30.

Further, the S26 includes: 5261, performing statistics on mean values and variances of R in all gSNP loci in 1q and 19p respectively and calculating a Z value of each R on chromosome 1 and chromosome 19 respectively based on 1q and 19p; S262, taking the mean values of Z values on 1q and 19p plus 2-6 times of variances respectively as threshold values of 1p and 19q; 5263, comparing the Z value of each gSNP locus on 1p and 19q with the corresponding threshold value to judge the LOH status of the locus, judging the LOH status of the locus abnormal if the Z value exceeds the threshold value, otherwise judging the LOH status of the locus normal; and S264, judging whether LOH occurs on 1p and 19q or not, performing statistics on numbers of abnormal and normal statuses on 1p and 19q respectively, judging that the LOH of the sample occurs on 1p/19q when abnormal statuses/(abnormal statuses+normal statuses) is greater than t₂, and judging co-deletion of 1p and 19q of the sample when only LOH occurs on 1p and 19q simultaneously, preferably, t₂ is greater than 0.6, and more preferably, t₂ is equal to 0.9.

Further, the S23 includes selecting the gSNP loci of the control sample in the first group of SNP loci according to a coverage and BAF, preferably, selection criteria of the gSNP loci are as follows: the coverage is greater than 100, the BAF range is 0.3-0.7.

Further, the public databases include SNP138, 1000 Genomes Project and Chinese Millionome Database, preferably, the selecting SNP locus includes selecting the loci SNP loci according to their allele frequency between 0.45-0.55 in the population; and preferably, an SNP locus being selected at every 200 kb.

Further, the detection method further includes a first verification step, the first verification step being used for detecting co-deletion of 1p and 19q based on STR and the first verification step comprising: S31, extracting known STR from existing data; S32, extracting reads covering the known STR from an alignment result file of the control sample, performing statistics on the number of repeat unit of the known STR on each read, extracting the two repeat units with the most numbers for each STR, marking as N₃ and N₄, wherein if

$$\frac{{N}_{3}}{{N}_{4}}$$

is greater than n, it is considered that the STR is a homozygotic type and is no longer configured for result judgment; and preferably, n is greater than 5, and more preferably, n is equal to 10; S33, extracting reads covering the known STR from an alignment result file of the to-be-tested sample, performing statistics the number of reads on the two repeat units determined in the control sample STR statistics module marking as T₃ and T₄ and calculating an LOH status of each STR, wherein the LOH status (Rⁱ) of the ith STR is defined as follows:

$${R}^{i} = \frac{{T}_{4}^{i} / {T}_{3}^{i}}{{N}_{4}^{i} / {N}_{3}^{i}} ;$$

and

S34, correcting R on 1p and 19q of the to-be-tested sample and determining a threshold value, judging the LOH status of each STR locus according to the threshold value and then judging co-deletion according to LOH statuses of all STR loci.

Further, the reads covering the known STR are reads covering from the upstream 20 bp to the downstream 20 bp of the known STR.

Further, the S34 includes: S341, judging the LOH status of each STR and judging that the LOH status of the locus is abnormal when R is smaller than T, otherwise, judging that the LOH status of the locus is normal, preferably, T is equal to 0.5; if R is greater than 1, converting to 1/R; and S342, judging whether LOH occurs on 1p and 19q or not, performing statistics on numbers of abnormal and normal statuses on 1p and 19q respectively, judging that the LOH of the sample occurs on 1p/19q when abnormal statuses/(abnormal statuses+normal statuses) is greater than t₃, and judging co-deletion of 1p and 19q of the sample when only LOH occurs on 1p and 19q simultaneously, preferably, t₃ is greater than 0.6, and more preferably, t₃ is equal to 0.8.

Further, the method further includes a second verification step, the second verification step being used for detecting co-deletion of 1p and 19q based on CNV.

Further, the method further includes sequencing data of MGMT gene promoter methylation, wherein the sequencing data of MGMT gene promoter methylation includes: acquiring methylated sequencing data originated from an MGMT gene promoter, wherein the methylated sequencing data is a double-end sequencing sequence; aligning the methylated sequencing data with a human reference genomic sequence to obtain an alignment result, the alignment result including a first end first matching region, a first end second matching region, a second end first matching region and a second end matching region, wherein the first end second matching region and the second end second matching region are overlapped; removing the first end second matching region or the second end second matching region in the alignment result to obtain to-be-analyzed data; and performing methylated locus recognition in the to-be-analyzed data to obtain a methylated result of the MGMT gene promoter.

Further, before aligning the methylated sequencing data with the human reference genomic sequence, sequencing data of MGMT gene promoter methylation further includes: pre-treatment of converting the human reference genomic sequence from C to T; and pre-treatment of converting the double-end sequencing sequence from C to T.

Further, after obtaining the to-be-analyzed data and before performing methylated loci recognition on the to-be-analyzed data, sequencing data of MGMT gene promoter methylation further includes a step of correcting the to-be-analyzed data, the step of correcting the to-be-analyzed data including: correcting the to-be-analyzed data by means of the human reference genomic sequence, position information of the human reference genomic sequence and high frequency SNP loci in a population.

Further, the step of performing methylated locus recognition in the to-be-analyzed data to obtain a methylated result of the MGMT gene promoter includes: identifying the methylated loci in the to-be-analyzed data primarily to obtain primarily identified loci; and selecting confidence of the primarily identified loci to obtain a methylated result of the MGMT gene promoter, preferably, criteria for the confidence selection are as follows: a coverage is smaller than 3000000, a possibility ratio standard between optimum and sub-optimum genotypes is greater than or equal to 20 and a comparison mass is greater than 5.

By applying the detection panel of the present disclosure to detect characteristic biomarkers, classificatory diagnosis and prognosis related genes, pharmacy related genes and cancer genesis and development related genes of glioma as well as polymorphic sites of effectiveness and toxic effects of a conventional chemotherapeutic scheme by combining NGS (also referred as to next generation sequencing), it is unnecessary to employ a plurality of experimental platforms and instruments and equipment, and the patient can be provided with precise and comprehensive diagnosis and treatment services merely by means of next generation sequencing. The cost is lowered greatly compared with that of a scheme in the prior art, and the detection panel is clinically popularized and applied.

### BRIEF DESCRIPTION OF THE DRAWINGS

The drawings of the description constituting a part of the present disclosure are to provide further understanding of the present disclosure. The schematic embodiment and description thereof are used for explaining the present disclosure and do not limit the present disclosure improperly. In the drawings,

FIG. 1 illustrates a flow schematic diagram of a processing method for sequencing data of MGMT gene promoter methylation according to the embodiment of the present disclosure;

FIG. 2 illustrates a schematic diagram of a processing device for sequencing data of MGMT gene promoter methylation according to the preferred embodiment of the present disclosure;

FIG. 3 and FIG. 4 respectively illustrate a schematic diagram of a FISH1p/19q detection result of a sample 1 in the embodiment 1 and a schematic diagram of a detection result of the method according to the embodiment;

FIG. 5 and FIG. 6 respectively illustrate a schematic diagram of a detection result of the method of the sample according to the embodiment 1 and a schematic diagram of a detection result of the one generation sequencing;

FIG. 7 and FIG. 8 respectively illustrate schematic diagrams of identifying three types of the same two sample results by employing the present disclosure in the embodiment 1;

FIG. 9 illustrates methylation level of each CpG locus detected by the pyrophosphate detection method in the embodiment 6; and

FIG. 10 illustrates methylation level of each CpG locus and methylation level on each DNA template molecule detected by the method of the present disclosure in the embodiment 6.

### DETAILED DESCRIPTION OF THE EMBODIMENTS

It should be noted that in the absence of conflict, the embodiments of the present disclosure and features in the embodiments can be combined with one another. The present disclosure will be described in detail below with reference to the accompanying drawings and the examples.

It should be noted that terms “First”, “second” and the like in the description, claims and drawings of the present disclosure are used for distinguishing similar objects and not have to describe a specific sequence or precedence order. It should be understood that data used in this way can be interchanged under a proper circumstance, facilitating the embodiments of the present disclosure described herein. In addition, terms “including”, “having”, and any variations thereof are intended to cover non-exclusive inclusions, for example, processes, methods, systems, products, or devices that includes a series of steps or units need not be limited to those clearly listed steps or units, but may include other steps or units not explicitly listed or inherent to these processes, methods, products, or devices.

In order to facilitate description, description on part of nouns or terms involved in the embodiments of the present disclosure is made below:

The forward and reverse strands of DNA mean two reverse complementary strands. The strand with reference to a genome is the so called forward strand while the other one is the reverse strand.

The sense strand and antisense strand mean that a set of strands carrying numbered protein information in two complementary DNAs is the sense strand which is also referred to as a coding strand, the sense strand is as same as a RNA sequence. The other strain complementary to the sense strain is referred to as the antisense strand. Although the antisense strand is complementary to RNA reversely, the antisense strand is the strand serving as a template of RNA, so that the antisense strand is also referred to as a template strand.

In a double-strand DNA molecule comprising a plurality of genes, the sense strands on each gene are not located on the same strand. That is, the sense strands of some genes are forward strands, the sense strands of other genes are reverse strands, i.e. one strand in the DNA double strands is, to some genes, the sense strand and is, to other genes, the antisense strand.

Chrom: chromosome number.

Loci: location.

R: LOH status ratio, deletion heterozygous status ratio.

R(LOH) is the LOH status ratio of each STR corresponding to the sample with 1p/19q co-deletion.

R(No LOH) is the LOH status ratio of each STR corresponding to the sample without 1p/19q co-deletion.

/ means that the STR locus is genetically homozygotic, which cannot be used for judgment.

Hom: homozygotic.

1p and 1q are a short arm of chromosome 1 and a long arm of chromosome 1 respectively.

19p and 19q are a short arm of chromosome 19 and a long arm of chromosome 19 respectively.

It is found by the applicants of the present disclosure that at present, representative molecular marker of glioma, diagnostic value, prognosis and predictive value and the corresponding detection method are shown as a table 1.

TABLE 1

| Molecular marker | Diagnostic value | Prognosis and predictive value | Detection method |
|---|---|---|---|
| IDH ½ mutation | / | Prognosis of the patent is good | IHC, sequencing |
| 1Pl9q deletion | Related to O-form | Prognosis of the patent is better | FISH, STR |
| | tightly | and radiotherapy/chemotherapy is | |
| | | recommended | |
| MGMT promoter | / | Radiotherapy/chemotherapy on a | IHC, |
| methylation | | patient with an anaplastic glioma | pyrosequencing |
| | | has a good curative effect | |
| EGFR amplification | Closely related to | Prognosis of GMB patients older | FISH |
| | GBM | than 60 years is poor | |
| EGFRVIII | Closely related to | Prognosis of GMB patients is | RT-PCR, IHC |
| rearrangement | GBM | poor | |
| PTEN mutation | / | Anaplastic astrocytoma prognosis | PCR, sequencing |
| | | related | |
| BRAF fusion | Closely related to | Targeted therapeutic targets | FISH, RT-PCR |
| | PA | | |
| BRAF point | PA | Targeted therapeutic targets | PCR, sequencing |
| mutation | | | |

Just as described in the background art, in conventional detection of glioma-related genes, it is necessary to combine a plurality of experimental platforms and instruments and equipment, and meanwhile, it is also necessary to purchase corresponding reagents. Many instruments and kits are equipped for finishing whole set of detection, and meanwhile, it is needed to operate each detection method by corresponding professionals, so that the general input cost is very high. In order to solve the technical problems, the present disclosure provides technical schemes as follows.

According to a typical implementation of the present disclosure, a next generation sequencing-based detection panel for glioma is provided. The detection panel includes glioma-related genes and loci. The glioma-related genes and loci includes SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPM1D, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR vIII, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1.

By applying the detection panel of the present disclosure to detect characteristic biomarkers, classificatory diagnosis and prognosis related genes, pharmacy related genes and cancer genesis and development related genes of glioma as well as polymorphic sites of effectiveness and toxic effects of a conventional chemotherapeutic scheme by combining NGS (also referred as to next generation sequencing), it is unnecessary to employ various experimental platforms and instruments and equipment, and the patient can be provided with precise and comprehensive diagnosis and treatment services merely by means of next generation sequencing. The cost is lowered greatly compared with that of a scheme in the prior art, and the detection panel is clinically popularized and applied.

In the typical implementation of the present disclosure, the detection data of the SNP locus on the chromosome 1 and the detection data of the SNP locus on the chromosome 19 can be analyzed by the following methods:

1) detecting all SNP loci on chromosome 1 and chromosome 19 in the control sample by publicly available SNP detection software;

2) selecting the gSNP loci of the control sample on the panel according to quality control parameters such as coverage and BAF, and performing statistics on numbers of reads of reference sequence genotypes (REF) and non-reference sequence genotypes (ALT), marked as N₁ and N₂, respectively. It is recommended that the BAF range is 0.3-0.7 and the coverage is greater than 100;

3) detecting all SNP loci on chromosome 1 and chromosome 19 of a to-be-tested tumor sample by publicly available SNP detection software;

4) performing statistics on numbers of reads of REF and ALT on gSNP determined in 2) according to the quality control parameters such as coverage, wherein the numbers are marked as T₁ and T₂ respectively and it is recommended that the coverage is greater than 100;

5) calculating the LOH status (deletion heterozygous status ratio) of each gSNP, wherein the LOH status ratio (Rⁱ) of the ith gSNP is defined as follows:

6)

$${R}^{i} = \vert \frac{\frac{{N}_{2}^{i}}{{N}_{1}^{i}}}{\frac{{N}_{2}^{i}}{{N}_{1}^{i}} + \frac{{T}_{2}^{i}}{{T}_{1}^{i}}} - 0.5 \vert$$

correcting R on 1q and 19p of the to-be-tested sample according to R of the gSNP loci on 1p and 19q of the to-be-tested sample and determining a threshold value, specifically including the steps of:

a. performing statistics on mean values and variances of R in all gSNP loci in 1q and 19p respectively and calculating a Z value of each R on chr1/chr19 respectively based on 1q/19p;

b. taking the mean values of Z values on 1q/19p plus four times of variances as 1p/19q threshold value, respectively,

c. comparing the Z value of each gSNP locus on 1p/19q with the corresponding threshold value to judge the LOH status of the locus, judging the LOH status of the locus abnormal if the Z value exceeds the threshold value, otherwise judging the LOH status of the locus normal;

judging whether LOH occurs on 1p/19q or not, performing statistics on numbers of abnormal and normal statuses on 1p/19q respectively, judging that LOH of the sample occurs on 1p/19q when abnormal statuses/(abnormal statuses+normal statuses) is greater than t, and judging co-deletion of 1p and 19q of the sample when only LOH occurs on 1p and 19q simultaneously, it is recommended that tis equal to 0.9.

By means of the method to correct information of 1p and 19q by combining information on 1q and 19p, the detection accuracy is improved, and 1p/19q co-deletion identification can be performed efficiently, conveniently and accurately.

Preferably, the glioma-related genes and loci further include STR locus on chromosome 1 and STR locus on chromosome 19. The detection result of the gene can be verified by means of data of the STR locus on chromosome 1 and data of the STR locus on chromosome 19.

For example, verification is carried out by the following steps:

extracting reads near the known STR from an alignment result file (bam file) of the control sample, and performing statistics on repetition times of known repetition units on each read: 1) extracting reads covering the known STR from an alignment result file of the control sample, performing statistics on the number of repeat unit of the known STR on each read, extracting the two repeat units with the most numbers for each STR, marked as N₃ and N₄; If

$$\frac{{N}_{3}}{{N}_{4}} > 10 ,$$

it is considered that the STR is homozygotic type, which is no longer configured for result judgment; performing statistics on read fully covering the whole STR region, wherein it is recommended that the coverage is greater than 100;

2) extracting reads near the known STR (reads of upstream 20 bp and downstream 20 bp of STR) from the alignment result file (bam file) of the to-be-tested sample, and performing statistics on repetition times of known repetition units on each read;

3) extracting reads cover the known STR from an alignment result file of the to-be-tested sample, performing statistics the number of reads on the two repeat units determined in 2), marked as T₃ and T₄; Recommending the read fully covering the whole STR region, wherein the coverage is greater than 100;

4) calculating the LOH status of each STR, wherein the LOH status (Rⁱ) of the Ith STR is defined as follows:

$${R}^{i} = \frac{{T}_{4}^{i} / {T}_{3}^{i}}{{N}_{4}^{i} / {N}_{3}^{i}} ;$$

5) judging the LOH status of each STR and judging that the LOH status of the locus is abnormal when R is smaller than T, otherwise, judging that the LOH status of the locus is normal; recommending T is equal to 0.5, and if R is greater than 1, converting to 1/R; 6) judging whether LOH occurs on 1p/19q or not, performing statistics on numbers of abnormal and normal statuses on 1p/19q respectively, judging that LOH of the sample occurs on 1p/19q when abnormal statuses/(abnormal statuses+normal statuses) is greater than t, and judging co-deletion of 1p and 19q of the sample when only LOH occurs on 1p and 19q simultaneously, recommending that t is equal to 0.8.

According to another aspect of the present disclosure, a next generation sequencing-based detection kit for glioma is provided. The detection kit includes a detection probe and/or a detection primer directed at glioma-related genes and loci, the glioma-related genes and loci including SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPM1D, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR vIII, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1. By applying the detection kit of the present disclosure to detect characteristic biomarkers, classificatory diagnosis and prognosis related genes, pharmacy related genes and cancer genesis and development related genes of glioma as well as polymorphic sites of effectiveness and toxic effects of a conventional chemotherapeutic scheme by combining NGS (also referred as to next generation sequencing), it is unnecessary to employ a plurality of experimental platforms and instruments and equipment, and the patient can be provided with precise and comprehensive diagnosis and treatment services merely by means of next generation sequencing. The cost is lowered greatly compared with that of a scheme in the prior art, and the detection panel is clinically popularized and applied.

Preferably, the glioma-related genes and loci further include STR locus on chromosome 1 and STR locus on chromosome 19. The detection result of the gene can be verified by data of the STR locus on chromosome 1 and data of the STR locus on chromosome 19.

Subjected to purpose of the present disclosure, according to the typical implementation of the present disclosure, the detection kit is used for detecting a plurality of mutation types, the plurality of mutation types including point mutation, fusion mutation, copy number variation, deletion mutation and insertion mutation. Preferably, the detection kit further includes primers for detecting the MGMT promoter methylation, wherein the primers for detecting the MGMT promoter methylation have sequences shown by SEQ ID NO: 1 and SEQ ID NO: 2. The primers are good in specificity and high in detection efficiency.

For the convenience of use, more preferably, the detection kit further includes one or more of groups consisting of DNA library construction reagents, gene trapping reagents, bisulfite conversion reagents and gene amplification reagents.

In order to improve the detection accuracy, the detection kit further includes a glioma panel verification sample, the glioma panel verification sample including IDH1, IDH2, TERT, ABL1, ALK, BRAF, EGFR, FGFR2, FLT3, GNA11, GNA11, GNAQ, JAK2, KIT, KRAS, MEK1, MET, NOTCH, NRAS, PDGFRA, PIK3CA and NTRK gene standard substances.

According to the typical implementation of the present disclosure, use of a next generation sequencing-based detection panel for glioma or the next generation sequencing-based detection kit for glioma in selecting drugs for treating or relieving glioma is provided. Preferably, the drug for treating or relieving glioma includes a targeted drug, a chemotherapeutic drug or an immune drug.

The next generation sequencing-based system for detecting 1p/19q co-deletion of glioma provided by the present disclosure is designed according to a principle that human body is a diploid creature, the mutation frequency (BAF, non-reference sequence genotype frequency) of heterozygous germline mutation is theoretically 50%, and actually, the finally obtained BAF fluctuates in a range near 50% due to various random factor influence in experiments. For the sample with positive LOH, the BAF of these SNP loci will be deviated from 50% due to DNA of tumor cells, and the higher the DNA concentration of the tumor cells in the to-be-tested sample is, the larger the deviation degree is. For the sample with negative LOH, the BAF near 50% can be stilled kept normally.

According to the typical implementation of the present disclosure, the next generation sequencing-based system for detecting 1p/19q co-deletion of glioma is provided. The system includes the SNP locus selection device, the SNP detection device without the control sample and/or the SNP detection device with the control sample, wherein the SNP locus selection device is configured to select the SNP loci on chromosome 1 and chromosome 19 according public databases to obtain a first group of SNP loci; the SNP detection device without the control sample includes: a first sequencing module configured for sequencing a to-be-tested sample and a group of negative samples; a first SNP detection module configured for detecting all SNP loci on chromosome 1 and chromosome 19 in the group of negative samples; a first gSNP locus selection module configured for selecting gSNP loci of the group of negative samples in the first group of SNP loci; a second SNP detection module configured for detecting all SNP loci on chromosome 1 and chromosome 19 in the to-be-tested sample; a first calculation and statistics module configured for performing calculation and statistics on BAF of mutated gSNP loci on the gSNP loci determined in the first gSNP loci selection module in the to-be-tested sample and marking the LOH status ratio (Rⁱ) of the ith gSNP as |BAF−0.5| of the ith gSNP; and a first judging module configured for correcting R on 1p and 19q of the to-be-tested sample according to R of the gSNP loci on 1q and 19p of the to-be-tested sample and determining a threshold value, judging the LOH status of each gSNP locus according to the threshold value and then judging co-deletion according to LOH statuses of all gSNP loci; the SNP detection device with the control sample includes: a second sequencing module configured for sequencing the to-be-tested sample and a control sample; a third SNP detection module configured for detecting all SNP loci on chromosome 1 and chromosome 19 in the control sample; a second gSNP locus selection module configured for selecting gSNP loci of the control sample in the first group of SNP loci; a fourth SNP detection module configured for detecting all SNP loci on chromosome 1 and chromosome 19 in the to-be-tested sample; a second calculation and statistics module configured for performing statistics on numbers of reads of reference sequence genotype and alternative sequence genotype of the control sample on the gSNP loci and marking as N₁ and N₂ respectively, performing statistics on numbers of reads of reference sequence genotype and alternative sequence genotype of the to-be-detected sample on the gSNP loci and marking as T₁ and T₂ respectively, calculating an LOH status ratio of each gSNP, wherein the LOH status (Rⁱ) of the ith gSNP is defined as follows: R

$${R}^{i} = \vert \frac{\frac{{N}_{2}^{i}}{{N}_{1}^{i}}}{\frac{{N}_{2}^{i}}{{N}_{1}^{i}} + \frac{{T}_{2}^{i}}{{T}_{1}^{i}}} - 0.5 \vert ;$$

and a second judging module configured for correcting R on 1p and 19q of the to-be-tested sample according to R of the gSNP loci on 1q and 19p of the to-be-tested sample and determining a threshold value, judging the LOH status of each gSNP locus according to the threshold value and then judging co-deletion according to LOH statuses of all gSNP loci.

By applying the technical scheme of the present disclosure to correct information of 1p and 19q by combining information on 1q and 19p, the detection accuracy is improved, and 1p/19q co-deletion identification can be performed efficiently, conveniently and accurately.

In a typical implementation of the present disclosure, the first judging module includes: a first statistics sub-module configured for performing statistics on mean values and variances of R in all gSNP loci in 1q and 19p respectively and calculating a Z value of each R on chromosome 1 and chromosome 19 respectively based on 1q and 19p; a first threshold value calculation sub-module configured for calculating the Z values of the group of negative samples corrected by 1q and 19p and taking the mth percentile as a threshold value, preferably, m is greater than 95, and more preferably, m is equal to 99; a first judging sub-module configured for comparing the Z value of each gSNP locus on 1p and 19q with the corresponding threshold value to judge the LOH status of the locus, judging the LOH status of the locus abnormal if the Z value exceeds the threshold value, otherwise judging the LOH status of the locus normal; and a second judging sub-module configured for judging whether LOH occurs on 1p and 19q or not, performing statistics on numbers of abnormal and normal statuses on 1p and 19q respectively, judging that the LOH of the sample occurs on 1p and 19q when abnormal statuses/(abnormal statuses+normal statuses) is greater than t₁, and judging co-deletion of 1p and 19q of the sample occurs when only LOH occurs on 1p and 19q simultaneously, preferably, t₁ is greater than 0.6, and more preferably, t₁ is equal to 0.8, the threshold value t₁ recommended in the present disclosure is an empirical value, so that a criteria for judging is neither strict excessively to lead to false negative nor to loose to lead to false positive, so that the judging accuracy is high.

Preferably, the first gSNP locus selection module selects the gSNP loci of the group of negative samples in the first group of SNP loci according to a coverage, BAF and a fluctuation size of BAF in the group of negative samples, preferably, selection criteria of the gSNP loci are as follows: a coverage is greater than 100, a BAF range is 0.1-0.9 and max-min of BAF among samples in the group of negative samples is smaller than 0.2; and preferably, the number of samples in the group of negative samples is greater than or equal to 30, thereby meeting a statistics effect.

In a typical implementation of the present disclosure, the second judging module includes: a second statistics sub-module configured for performing statistics on mean values and variances of R in all gSNP loci in 1q and 19p respectively and calculating a Z value of each R on chromosome 1 and chromosome 19 respectively based on 1q and 19p; a second threshold value calculation sub-module configured for taking the mean values of Z values on 1q and 19p plus 2-6 times of variances respectively as threshold values of 1p and 19q; a third judging sub-module configured for comparing the Z value of each gSNP locus on 1p and 19q with the corresponding threshold value to judge an LOH status of the locus, judging the LOH status of the locus is abnormal if the Z value exceeds the threshold value, otherwise judging the LOH status of the locus is normal; and a fourth judging sub-module configured for judging whether LOH occurs on 1p and 19q or not, performing statistics on numbers of abnormal and normal statuses on 1p and 19q respectively, judging that the LOH of the sample occurs on 1p/19q when abnormal statuses/(abnormal statuses+normal statuses) is greater than t₂, and judging co-deletion of 1p and 19q of the sample occurs when only LOH occurs on 1p and 19q simultaneously, preferably, t₂ is greater than 0.6, and more preferably, t₂ is equal to 0.9. The threshold value t₂ recommended in the present disclosure is an empirical value, so that a condition for judging is neither strict excessively to lead to false negative nor to loose to lead to false positive, so that the judging accuracy is high.

Preferably, the second gSNP locus selection module selects the gSNP loci of the control sample in the first group of SNP loci according to a coverage and BAF, preferably, selection criteria of the gSNP loci are as follows: a coverage is greater than 100, and a BAF range is 0.3-0.7. the threshold value BAF recommended in the present disclosure is an empirical value, so that a condition for judging is neither strict excessively to lead to false negative nor to loose to lead to false positive, so that the judging accuracy is high.

Preferably, the public databases include SNP138, 1000 Genomes Project and Chinese Millionome Database, preferably, the SNP locus selection device selecting the loci SNP loci according to their allele frequency 0.45-0.55 in the population; and preferably, an SNP locus being selected at every 200 kb.

In a typical implementation of the present disclosure, the system includes a first verification device, the first verification device being configured to detect co-deletion of 1P and 19q based on STR and the first verification device including: an STR acquisition module configured for extracting known STR from existing data; a control sample STR statistics module configured for extracting reads covering the known STR from an alignment result file of the control sample, performing statistics on the number of repeat unit of the known STR on each read, extracting the two repeat units with the most numbers for each STR, marking as N₃ and N₄, wherein if

$$\frac{{N}_{3}}{{N}_{4}}$$

is greater than n, it is considered that the STR is a homozygotic type and is no longer configured for result judgment; and preferably, n is greater than 5, and more preferably, n is equal to 10; a to-be-tested sample STR statistics module configured for extracting reads covering the known STR from an alignment result file of the to-be-tested sample, performing statistics the number of reads on the two repeat units determined in the control sample STR statistics module marking as T₃ and T₄ and calculating an LOH status of each STR, wherein the LOH status (Rⁱ) of the ith STR is defined as follows:

$${R}^{i} = \frac{{T}_{4}^{i} / {T}_{3}^{i}}{{N}_{4}^{i} / {N}_{3}^{i}} ;$$

and a third judging module configured for correcting R on 1q and 19p of the to-be-tested sample determining a threshold value, judging the LOH status of each STR locus according to the threshold value and then judging co-deletion according to LOH statuses of all STR loci; preferably, the reads covering the known STR are reads covering from the upstream 20 bp to the downstream 20 bp of the known STR. Thus, a condition that the operating time is increased as the sequencing sequence is too long is avoided or a condition that less read sequences are extracted as the sequencing sequence is too short is avoided. Preferably, the third judging module includes: a fifth judging sub-module configured for judging the LOH status of each STR and judging that the LOH status of the locus is abnormal when R is smaller than T, otherwise, judging that the LOH status of the locus is normal, preferably, T is equal to 0.5; if R is greater than 1, converting to 1/R; a sixth judging sub-module configured for judging whether LOH occurs on 1p and 19q or not, performing statistics on numbers of abnormal and normal statuses on 1p and 19q respectively, judging that the LOH of the sample occurs on 1p/19q when abnormal statuses/(abnormal statuses+normal statuses) is greater than t₃, and judging co-deletion of 1p and 19q of the sample when only LOH occurs on 1p and 19q simultaneously, preferably, t₃ is greater than 0.6, and more preferably, t₃ is equal to 0.8. the threshold value t recommended in the present disclosure is an empirical value, so that a criterion for judging is neither strict excessively to lead to false negative nor to loose to lead to false positive, so that the judging accuracy is high. Preferably, the system includes a second verification device, the second verification device being configured to detect co-deletion of 1p and 19q based on CNV.

According to the typical embodiment (embodiment 1) of the present disclosure, the next generation sequencing-based system for detecting co-deletion of 1p/19q of glioma executes the following method actually:

1. Panel Design

1) selecting a public database SNP138, a 1000 Genomes Project and a Chinese Millionome Database and an internal database, and selecting the loci according to the frequency range of minimum allelic mutation in the population. Recommended range: 0.45-0.55.

2) considering distributing uniformity of the whole chromosome arms 1 and 19, and selecting a SNP locus at every 200 kb.

3) finally, selecting 814 SNPs meeting the criteria on the chromosome 1 and chromosome 19, including 325 loci on the short arm (1p) of the chromosome 1 and the long arm (19q) of the chromosome 19.

4) combining records of published literature, the design includes 17 short tandem repeat sequences (STR) regions, 11 STR regions on 1p and 6 STR regions on 19q.

2. SNP-Based Identification Method for Co-Deletion of 1p and 19q

2.1 With a Control Sample

Based on the principle described therein, the first step is to search for germline heterozygous mutation (gSNP) of the to-be-tested sample. As different to-be-tested samples are different in gSNP, using the control sample aims to determine the gSNP accurately.

7) detecting all SNP loci on chromosome 1 and chromosome 19 in the control sample by publicly available SNP detection software;

8) selecting the gSNP loci of the control sample on the panel according to quality control parameters such as coverage and BAF, and performing statistics on numbers of reads of reference sequence genotypes (REF) and non-reference sequence genotypes (ALT), marked as N₁ and N₂, respectively. It is recommended that the BAF range is 0.3-0.7 and the coverage is greater than 100;

9) detecting all SNP loci on chromosome 1 and chromosome 19 of a to-be-tested tumor sample by publicly available SNP detection software;

10) performing statistics on numbers of reads of REF and ALT on gSNP determined in 2) according to the quality control parameters such as coverage, wherein the numbers are marked as T₁ and T₂ respectively. Recommending the coverage is greater than 100;

11) calculating the LOH status of each gSNP, wherein the LOH status (R¹) of the ith gSNP is defined as follows:

12)

$${R}^{i} = \vert \frac{\frac{{N}_{2}^{i}}{{N}_{1}^{i}}}{\frac{{N}_{2}^{i}}{{N}_{1}^{i}} + \frac{{T}_{2}^{i}}{{T}_{1}^{i}}} - 0.5 \vert$$

correcting R on 1q and 19p of the to-be-tested sample according to R of the gSNP loci on 1p and 19q of the to-be-tested sample and determining a threshold value, specifically including the steps of:

d. performing statistics on mean values and variances of R in all gSNP loci in 1q and 19p respectively and calculating a Z value of each R on chr1/chr19 respectively based on 1q/19p;

e. taking the mean values of Z values on 1q/19p plus four times of variances as 1p/19q threshold value, respectively;

f. comparing the Z value of each gSNP locus on 1p/19q with the corresponding threshold value to judge the LOH status of the locus, judging the LOH status of the locus is abnormal if the Z value exceeds the threshold value, otherwise judging the LOH status of the locus is normal;

13) judging whether LOH occurs on 1p/19q or not, performing statistics on numbers of abnormal and normal statuses on 1p/19q respectively, judging that LOH of the sample occurs on 1p/19q when abnormal statuses/(abnormal statuses+normal statuses) is greater than t₂, and judging co-deletion of 1p and 19q of the sample when only LOH occurs on 1p and 19q simultaneously, it is recommended that t₂ is equal to 0.9.

2.2 Without a Control Sample

Sometimes, as it is limited to draw materials of samples, control samples corresponding to the to-be-tested samples are not found always, so that the patent further provides a SNP-based identification method for co-deletion of 1p and 19q without a control sample.

1) preparing a group of negative samples and detecting all SNP loci on chromosome 1 and chromosome 19 in the group of samples by publicly available SNP detection software, and it is recommended that n is equal to 30;

2) selecting the gSNP loci of the groups of samples on the panel according to the quality control parameters such as coverage, BAF and fluctuation amplitude of BAF in the groups of samples. Recommending that the BAF range is 0.1-0.9, the coverage is greater than 100, and max-min of BAF among samples in the group of negative samples is smaller than 0.2;

3) detecting all SNP loci on chromosome 1 and chromosome 19 of a to-be-tested tumor sample by publicly available SNP detection software;

4) performing statistics on gSNP determined in 2) and BAF with mutation of the to-be-tested sample according to the quality control parameters such as coverage, and it is recommended that the coverage is greater than 100;

5) calculating the LOH status of each gSNP, wherein the LOH status ratio (Rⁱ) at the location is |BAF−0.5|;

6) correcting R on 1q and 19p of the to-be-tested sample according to R of the gSNP loci on 1p and 19q of the to-be-tested sample and determining a threshold value, specifically including the steps of:

a. performing statistics on mean values and variances of R in all gSNP loci in 1q and 19p respectively and calculating a Z value of each R on chr1/chr19 respectively based on 1q/19p;

b. calculating the Z values of the group of negative samples corrected by 1q and 19p and taking the mth percentile as a threshold value, recommending that m is equal to 99; and

c. comparing the Z value of each gSNP locus on 1p/19q with the corresponding threshold value to judge the LOH status of the locus, judging the LOH status of the locus is abnormal if the Z value exceeds the threshold value, otherwise judging the LOH status of the locus is normal;

7) judging whether LOH occurs on 1p/19q or not, performing statistics on numbers of abnormal and normal statuses on 1p/19q respectively, judging that LOH of the sample occurs on 1p/19q when abnormal statuses/(abnormal statuses+normal statuses) is greater than t₁, and judging co-deletion of 1p and 19q of the sample when only LOH occurs on 1p and 19q simultaneously, it is recommended that t₁ is equal to 0.8.

3. STR-Based Identification Method for Co-Deletion of 1p and 19q

3.1 With a Control Sample

7) extracting reads near the known STR from an alignment result file (bam file) of the control sample, and performing statistics on repetition times of known repetition units on each read:

8) performing statistics on the number of repeat unit of the known STR on each read, extracting the two repeat units with the most numbers for each STR, marking as N₃ and N₄; If

$$\frac{{N}_{3}}{{N}_{4}} > 10 ,$$

it is considered that the STR is homozygotic type, which is no longer configured for result judgment; performing statistics on read fully covering the whole STR region, wherein it is recommended that the coverage is greater than 100;

9) extracting reads near the known STR (reads of upstream 20 bp and downstream 20 bp of STR) from the alignment result file (bam file) of the to-be-tested sample, and performing statistics on repetition times of known repetition units on each read;

10) extracting reads cover the known STR from an alignment result file of the to-be-tested sample, performing statistics the number of reads on the two repeat units determined in 2), marked as T₃ and T₄; and it is recommended that the read fully covering the whole STR region, wherein the coverage is greater than 100;

11) calculating the LOH status of each STR, wherein the LOH status (Rⁱ) of the ith STR is defined as follows:

$${R}^{i} = \frac{{T}_{4}^{i} / {T}_{3}^{i}}{{N}_{4}^{i} / {N}_{3}^{i}} ;$$

12) judging the LOH status of each STR and judging that the LOH status of the locus is abnormal when R is smaller than T, otherwise, judging that LOH status of the locus is normal; and it is recommended that T is equal to 0.5, and if R is greater than 1, converting to 1/R;

13) judging whether LOH occurs on 1p/19q or not, performing statistics on numbers of abnormal and normal statuses on 1p/19q respectively, judging that LOH of the sample occurs on 1p/19q when abnormal statuses/(abnormal statuses+normal statuses) is greater than t₃, and judging co-deletion of 1p and 19q of the sample when only LOH occurs on 1p and 19q simultaneously, and it is recommended that t₃ is equal to 0.8.

4. CNV-Based Identification Method for Co-Deletion of 1p and 19q

In some cases, co-deletion of 1p and 19q means that the copy numbers on 1p and 19q are no longer 2 but is turned to 1. From a CNV result, if loss (LOSS) occurs on the whole chromosome arms 1q and 19q, it is judged that co-deletion of 1p and 19q occurs.

The CNV result on 1p and 19q is detected by employing the publicly available CNV detection software. It is recommended that the ctCNV method (publication number CN108319813A) disclosed by the team is used.

The method includes the main steps of:

1) acquiring a group (n) of alignment result files (it is recommended that n is greater than 30) between a normal control crowd sample and the to-be-tested sample and a human reference genome;

2) performing standardization for the number of reads on the targeted region according to data size, GC content and a captured region length;

3) extracting the normal control crowd alignment result files to establish a base line and calculating the fluctuation range of a healthy person with different genome levels and statistics scores; and

4) calculating the change multiple and statistics scores of CNV of the to-be-tested sample compared with the crowd base line, judging significance and outputting the copy numbers.

As mentioned in the background art, in the prior art, there is a defect that the detection method of MGMT gene promoter methylation is low in efficiency or low in accuracy. In order to improve the condition, the inventors perform comparative analysis on the public detection method of MGMT gene promoter methylation, and find that the CG content and the TM value in a sequence region will be variable to a great extend as part of C base will be converted into T after a DNA sequence is treated by sulfite when the primer is designed by the existing hydrosulphite sequencing RCR (BSP) method, thereby further affecting acquisition of an ideal primer sequence by conventional primer design software on the sequence. In order to provide an amplification primer better in specificity and higher in amplification efficiency, the inventors have designed dozens of pairs of primers for the gene promoter loci, and selected candidate targeted primers by simulating the GC content and the TM value after the C base are converted into T by considering the characteristics of DNA treated by sulfite fully. Further, through experimental verification, a pair of primers with best amplification efficiency and specificity is determined finally. It is attempted to perform methylation detection by the NGS method based on an amplified product of the primer. It is found by the sequencing data through an improved methylation analytical flow that not only is the accuracy of finally detected methylated loci high, but also is the throughput of the detected loci high correspondingly. Therefore, it is convenient to evaluate the methylation level by combining the integral methylated locus information.

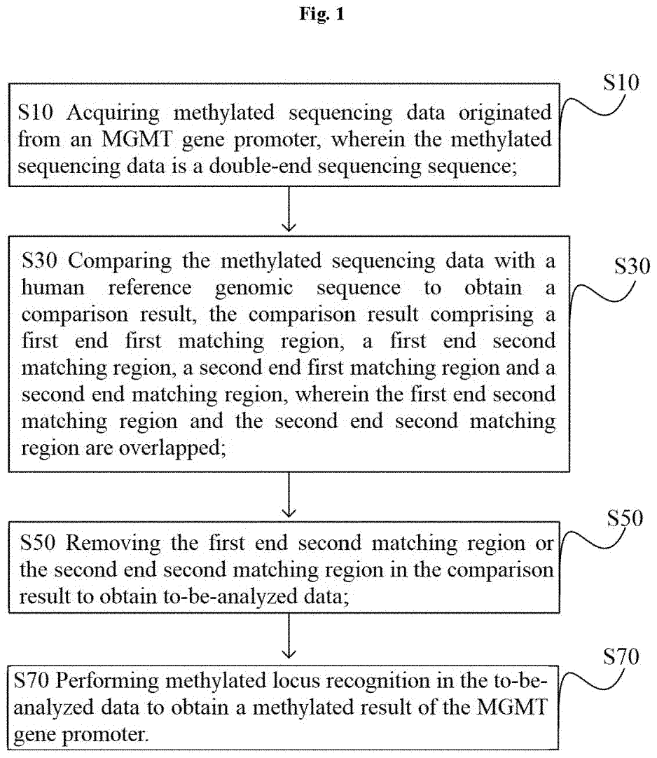

Based on the research result, the applicants provide the technical scheme of the present disclosure. In a typical implementation, a processing method for sequencing data of MGMT gene promoter methylation is provided. FIG. 1 illustrates the flow diagram of the processing method for sequencing data of MGMT gene promoter methylation in the implementation of the present disclosure. As shown in the FIG. 1, the processing method includes:

S10. acquiring methylated sequencing data originated from an MGMT gene promoter, wherein the methylated sequencing data is a double-end sequencing sequence;

S30. aligning the methylated sequencing data with a human reference genomic sequence to obtain an alignment result, the alignment result comprising a first end first matching region, a first end second matching region, a second end first matching region and a second end matching region, wherein the first end second matching region and the second end second matching region are overlapped;

S50. removing the first end second matching region or the second end second matching region in the alignment result to obtain to-be-analyzed data; and

S70. performing methylated locus recognition in the to-be-analyzed data to obtain a methylated result of the MGMT gene promoter.

According to the processing method for sequencing data of MGMT gene promoter methylation, by aligning sequencing data at two ends in the alignment result with sequences in the overlapped region to remove repetition, the result in follow-up recognition and statistics of methylation level is more accurate.

In the aligning step, an existing methylation alignment policy is adopted. In a preferred implementation, before aligning the methylated sequencing data with the human reference genomic sequence, the processing method further includes: pre-treatment of converting the human reference genomic sequence from C to T; and pre-treatment of converting the double-end sequencing sequence from C to T.

Specifically, according to an amplification source (originated from either a forward strand or a reverse strand of a genome) of the to-be-processed methylated sequencing data, after conversion pre-treatment from C to T (or G to A) respectively, the sense strand and the antisense strand corresponding to the forward strand or the reverse strand of the corresponding human reference genomic sequence are taken as reference alignment sequences. Correspondingly, conversion pre-treatment from C to T (or from G to A) is carried out on the sequencing sequence at each end in the double-end sequencing sequence respectively.

Before alignment, it is not clear that the double-end sequencing sequence is the forward strand or the reverse strand of the human reference genomic sequence. It can be determined only after alignment according to an alignment position.

In order to make the follow-up methylation level of each locus more accurate relatively, in a preferred implementation, after obtaining the to-be-analyzed data and before performing methylated loci recognition on the to-be-analyzed data, the processing method further includes a step of correcting the to-be-analyzed data, the step of correcting the to-be-analyzed data including: correcting the to-be-analyzed data by means of the human reference genomic sequence, position information of the human reference genomic sequence and high frequency SNP loci in a population.

The correcting step can remove some loci with poor quality, the quality including sequencing quality or alignment quality. Specific correcting software can perform correction by adopting a Bisulfite Count Covariates module and a Bisulfite Table Recalibration module in BisSNP software. It is favorable to improve the identification accuracy by carrying out the correcting step.

In order to further improve the confidence of each methylated locus, in a preferred implementation, the step of performing methylated locus recognition in the to-be-analyzed data to obtain a methylated result of the MGMT gene promoter includes: identifying the methylated loci in the to-be-analyzed data primarily to obtain primarily identified loci; and selecting confidence of the primarily identified loci to obtain a methylated result of the MGMT gene promoter, preferably, criteria for the confidence selection are as follows: a coverage is smaller than 3000000, a possibility ratio standard between optimum and sub-optimum genotypes is greater than or equal to 20 and a comparison mass is greater than 5.

Specifically, the primary identifying step can be used for identifying SNP/methylated loci simultaneously by adopting a Bisulfite Genotyper module of BisSNP to obtain initial vcf files of methylation of SNP and CpG, respectively. The primarily identified methylated vcf files are sequenced according to genomic positions through a sort By Ref And Cor module of BisSNP and then methylated loci with low confidence in the sequenced methylated vcf files are filtered by adopting a VCF post process module of BisSNP. A specific filter criterion adopts default values of the software module.

It should be noted that the step illustrated by the flow diagram can be executed in, for example, a computer system with a group of computer executable commands, and furthermore, although a logic sequence is shown in the flow diagram, under some circumstances, the illustrated or described steps can be executed in a sequence different from that herein.

The implementation of the present disclosure further provides a processing device for sequencing data of MGMT gene promoter methylation. It should be noted that the processing device of the implementation of the present disclosure can be used for executing the processing method for sequencing data of MGMT gene promoter methylation in the implementation of the present disclosure. The processing device is introduced below.

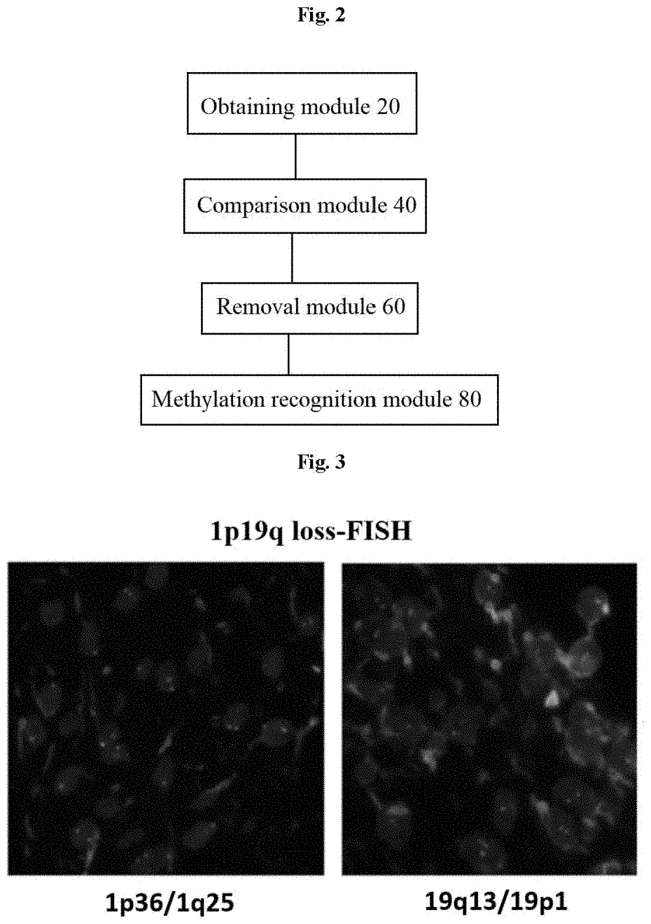

FIG. 2 illustrates a schematic diagram of a processing device for sequencing data of MGMT gene promoter methylation according to the implementation of the present disclosure. As shown in the FIG. 2, the processing device includes an acquisition module 20, an alignment module 40, a removal module 60 and a methylation recognition module 80.

The acquisition module 20 is configured to acquire methylated sequencing data originated from an MGMT gene promoter, wherein the methylated sequencing data is a double-end sequencing sequence;

the alignment module 40 is configured to align the methylated sequencing data with a human reference genomic sequence to obtain an alignment result, the alignment result comprising a first end first matching region, a first end second matching region, a second end first matching region and a second end matching region, wherein the first end second matching region and the second end second matching region are overlapped;

the removal module 60 is configured to remove the first end second matching region or the second end second matching region in the alignment result to obtain to-be-analyzed data; and

the methylation recognition module 80 is configured for methylated locus recognition in the to-be-analyzed data to obtain a methylated result of the MGMT gene promoter.

The processing device acquires the methylated sequencing data of a targeted fragment through the acquisition module, then executes the alignment module to obtain an alignment result and then executes the removal module to remove repetition by aligning sequencing data at two ends in the alignment result with sequences in the overlapped region, so that the result in follow-up recognition and statistics of methylation level is more accurate.

The alignment module can adopt an existing methylated alignment module. In a preferred implementation, the processing device further includes: a first pre-treatment module configured for pre-treatment of converting C of the human reference genomic sequence to T; and a second pre-treatment module configured for pre-treatment of converting C of the double-end sequencing sequence to T.

Specifically, according to an amplification source (originated from either a forward strand or a reverse strand of a genome) of the to-be-processed methylated sequencing data, after conversion pre-treatment from C to T (or G to A) respectively, the sense strand and the antisense strand corresponding to the forward strand or the reverse strand of the corresponding human reference genomic sequence are taken as reference alignment sequences. Correspondingly, conversion pre-treatment from C to T (or from G to A) is carried out on the sequencing sequence at each end in the double-end sequencing sequence respectively.

Before alignment, it is not clear that the double-end sequencing sequence is the forward strand or the reverse strand of the human reference genomic sequence. It can be determined only after alignment according to an alignment position.

In order to make the follow-up methylation level of each locus more accurate relatively, in a preferred implementation, the processing device further includes a correction module configured for correcting the to-be-analyzed data, the correction module being configured to correct the to-be-analyzed data by means of the human reference genomic sequence, position information of the human reference genomic sequence and high frequency SNP loci in a population.

The correction module can remove some loci with poor quality, the quality including sequencing quality or alignment quality. Specific correcting software can perform correction by adopting a Bisulfite Count Covariates module and a Bisulfite Table Recalibration module in BisSNP software. It is favorable to improve the identification accuracy by carrying out the correction module.

In order to further improve the confidence of each methylated locus, in a preferred implementation, the methylated recognition module includes: a primary identification module configured for identifying the methylated loci in the to-be-analyzed data primarily to obtain primarily identified loci; and a confidence selection module configured for confidence selection of the primarily identified loci to obtain a methylated result of the MGMT gene promoter, preferably, criteria for the confidence selection are as follows: a coverage is smaller than 3000000, a possibility ratio standard between optimum and sub-optimum genotypes is greater than or equal to 20 and a comparison mass is greater than 5.

In a third typical implementation, a method for detecting MGMT gene promoter methylation is provided. The method includes carrying out bisulfite conversion on gDNA of the to-be-tested sample to obtain converted DNA; constructing an amplicon library with the converted DNA to obtain the amplicon library; sequencing the amplicon library to obtain sequencing data; and carrying out methylated analysis on the sequencing data by adopting any one of processing method or processing device to obtain the methylated result information of MGMT gene promote methylation.

According to the detection method of the present disclosure, the processing flow of the methylated sequencing data is adopted, so that the detection result of MGMT gene promoter methylation is more accurate.

The amplification primer of the targeted gene promoter of the present disclosure is improved, so that based on better amplification efficiency and specificity, the detection method of the prevent disclosure further includes an improved amplicon library construction scheme. In a preferred implementation, the amplicon library is constructed with the converted DNA by adopting the amplification primer to obtain the amplicon library, wherein the amplification primer includes an upstream sequence and a downstream sequence, the upstream sequence being SEQ ID NO: 1 and the downstream sequence being SEQ ID NO: 2.

The detection method provided by the present disclosure is not only high in amplification efficiency, but also high in specificity by amplifying the targeted region by using the improved primer, and thus, the DNA information of the targeted region is relatively more accurate. Further, the targeted region is amplified to the amplicon library, and the methylation condition is detected by the high throughput sequencing method, so that the quantity of the methylated loci of MGMT gene promoter is improved, i.e. the detecting throughput and efficiency are improved.

In order to amplify the promoter region of the targeted gene more efficiently, the inventors further optimize the working concentration and the annealing temperature of the designed primer, so that the amplification efficiency and specificity are improved. Thus, in a preferred implementation, the working concentration of the primer is 5-15 μM, preferably 10 μM. In another preferred implementation, the annealing temperature of the primer in a amplification process is 45-55° C., preferably 50° C. In some other preferred implementations, in the process of constructing the amplicon library with the converted DNA by the amplified primer, 30-40 cycles are amplified for the converted DNA, preferably 35 cycles, to obtain the amplicon library.

The beneficial effects of the present disclosure will be further described below in combination of the implementations. In the implementations, steps or reagents not described in detail can be realized by using conventional operations or conventional commercial reagents in the field without affecting the final result of the present disclosure substantially.

### Embodiment 1

(I) Breaking, Library Constructing and Capturing Steps for a FFPE Sample

I, Preparation of a Glioma Panel Verification Sample

standard substance: In the experiment, glioma-related genes such as IDH1, IDH2, TERT, ABL1, ALK, BRAF, EGFR, FGFR2, FLT3, GNA11, GNA11, GNAQ, JAK2, KIT, KRAS, MEK1, MET, NOTCH, NRAS, PDGFRA, PIK3CA, NTRK are selected to configure 18 standard substances with different mutation frequencies, performance analysis is carried out from three aspects: variation of copy number, rearrangement and point mutation through the steps of breaking, library construction and loading after capturing and enriching and biological information analysis.

Clinical samples: 37 pairs of glioma samples verified by other methods are selected, and performance analysis is carried out from three aspects: variation of copy number, rearrangement and point mutation through the steps of library construction and loading after capturing and enriching and biological information analysis.

II Tissue DNA Extraction and Breaking:

Tissue DNA is extracted by using a tissue DNA extraction kit. The extracted DNA is quantified by using Qubit 3.0 and dsDNA HS Assay Kit.

A polytetrafluoroethylene line is cut to a length about 1 cm by using a pair of ultraviolet sterilized medical scissors, and it is ensured that the length uniformity of a breaking bar is good, and the polytetrafluoroethylene line is put in a clean container for ultraviolet sterilization for 3-4 hours. After sterilization, the 1 cm polytetrafluoroethylene line is fed into a 96 porous plate by using a sterilized embedder. Two breaking bars are contained in each hole, and the 96 porous plate is sterilized by ultraviolet rays for 3-4 hours after the breaking bars are contained in each hole.

300 ng of tissue DNA sample is fetched according to a qubit quantified result, is diluted to 50 μl by using a TE diluent and is transferred to the 96 porous plate. A tin foil paper film is placed on the 96 porous plate with four edges aligned, the film is sealed twice by using a hot film sealer for 5 s at 180° C., and the film is centrifugalized by using a micro-porous plate centrifugal machine.

Select the preset program Peak Power: 450, Duty Factor: 30, Cycles/Burst: 200, Treatment time: 40 s, 3 cycles and click “Start position”. Click “Run” button at a Run interface to operate the program. After the program is operated, a sample plate is taken out, centrifugalized by using the micro-porous plate centrifugal machine and placed on a sample rack. Select the program Peak Power: 450, DutyFactor: 30, Cycles/Burst: 200, Treatment time: 40 s, 4 cycles. Click “Run” button at a Run interface to operate the program. After the program is operated, the sample plate is taken out and centrifugalized by using the micro-porous plate centrifugal machine. 1 μl of the sample is taken after being broken for quality inspection.

III Library Construction

1. End Repair and Addition of Tail a at 3′ End:

1.1. 50 μL of DNA is taken, supplementing the DNA to 50 μL with water without nuclease if the DNA is not 50 μL, and added into a reaction system according to the following table 2:

TABLE 2

| Component | Volume |
|---|---|
| End repair and addition of buffer solution A | 7 μL |
| End repair and addition of enzyme A | 3 μL |
| DNA | 50 μL |
| Total volume | 60 μL |

1.2 DNA is mixed uniformly by vortex, micro-centrifugalized and placed in a PCR instrument, and the reaction program is as shown in the following table 3.

TABLE 3

| Step | Temperature | time |
|---|---|---|
| End repair and addition of A | 20° C. | 30 min |
| | 65° C. | 30 min |
| termination | 20° C. | ∞ |

2. Connecting Adapter:

2.1 Adapter preparation: 2.5 μL of adapter is added with 2.5 μL of water to be diluted to 5 μL.

2.2 Corresponding reagents are added into a reaction tube according to the following table 4:

TABLE 4

| Component | Volume |
|---|---|
| Water without nuclease | 5 μL |
| Connecting buffer solution | 30 μL |
| DNA ligase | 10 μL |
| End repair and addition of reaction product A | 60 μL |
| Total volume | 110 μL |

2.3 DNA is mixed uniformly by vortex, micro-centrifugalized and placed in a PCR instrument, and the reaction program is as shown in the following table 5:

TABLE 5

| Step | Temperature | time |
|---|---|---|
| Adapter connection | 20° C. | 30 min |
| termination | 20° C. | ∞ |

Note: lid temperature is 50° C.

3. Purification after Connection:

3.1 Beckman Agencourt AMPure XP magnetic beads are sub-packaged to a new 8-tube strip, 88 μL in each. After PCR in the previous step is finished, 2.3 is finished, the sample is taken out, centrifugalized transiently and transferred to the sub-packaged 88 μL magnetic bead centrifugal tube.

3.2 The mixture is vibrated and mixed uniformly, and incubated for 15 min at room temperature, so that DNA and the magnetic beads are combined fully. Note: a pipe cover is pressed during vibration. The mixture is centrifugalized transiently, a centrifuge tube is placed on a magnetic rack to clarify a liquid and a supernatant is removed (it is ensured that the residual amount does not exceed 5 μL). Note: do not absorb the magnetic beads.

3.3 200 μL of 80% ethanol is added to incubate for 30 see to be abandoned. The cleaning step by 200 μL of 80% ethanol is repeated once. Note: 80% ethanol is prepared when it is in need.

3.4 Residual ethanol at the bottom of the centrifugal tube is absorbed thoroughly with a 10 μL pipette tip and is dried for 3-5 min at room temperature till ethanol is volatilized fully (no reflective light on the front surface and the back surface has been dried). Note: the DNA output will be reduced if the magnetic beads are dried excessively.

3.5 The centrifuge tube is taken down from the magnetic rack and 21 μL of ultrapure water is added for vibration and uniform mixing. Note: a pipe cover is pressed during vibration. Incubated at room temperature for 5 min.

3.6 The mixture is centrifugalized transiently, the centrifuge tube is placed on the magnetic rack to clarify the liquid. The residual 20 μL of supernate is transferred to a new PCR tube for a next step amplification test.

4. Library Amplification:

4.1 Adding to the reaction system according to the following table 6:

TABLE 6

| Component | Volume |
|---|---|
| Hot start enzyme | 25 μL |
| Mixture of primer and reaction buffer solution | 5 μL |
| Adapter connection product | 20 μL |
| Total volume | 50 μL |

4.2 DNA is mixed uniformly by vortex, micro-centrifugalized and placed in a PCR instrument, and the reaction program is as shown in the following table 7:

TABLE 7

| Step | Temperature | time | Cycle number |
|---|---|---|---|
| Predegeneration | 98° C. | 45 sec | |
| Degeneration | 98° C. | 15 sec | Determined according |
| Annealing | 60° C. | 30 sec | to concentration |
| Extending | 72° C. | 30 sec | |
| Re-extending | 72° C. | 1 min | |
| Save | 8° C. | ∞ | |

5. Acquisition of DNA

5.1 25 μL of Beckman Agencourt AMPure XP magnetic beads are sub-packaged to a new 8-tube strip.

5.2 After PCR in the previous step (4.2) is finished, the sample is taken out.

5.3 The mixture is centrifugalized transiently and is transferred to 25 μL of sub-packaged Beckman Agencourt AMPure XP magnetic beads.

5.4 The mixture is vibrated and mixed uniformly, and is incubated for 15 min at room temperature, so that DNA and the magnetic beads are combined fully. Note: the tube cover is pressed during vibration.

5.5 The mixture is centrifugalized transiently, the centrifuge tube is placed on the magnetic rack to clarify the liquid, and the supernatant is transferred to the other tube of sub-packaged Beckman Agencourt AMPure XP magnetic beads. Note: do not absorb the magnetic beads.

5.6 The mixture is vibrated and mixed uniformly, and is incubated for 15 min at room temperature, so that DNA and the magnetic beads are combined fully. Note: a pipe cover is pressed during vibration.

5.7 The mixture is centrifugalized transiently, the centrifuge tube is placed on the magnetic rack to clarify the liquid, and the supernatant is abandoned. Note: do not absorb the magnetic beads.

5.8 200 μL of 80% ethanol is added to incubate for 30 sec to be abandoned. Note: 80% ethanol is prepared when it is in need. The cleaning step by 200 μL of 80% ethanol is repeated once.

5.9 Residual ethanol at the bottom of the centrifugal tube is absorbed thoroughly with a 10 μL pipette tip and is dried for 3-5 min at room temperature till ethanol is volatilized fully (no reflective light on the front surface and the back surface has been dried). Note: the DNA output will be reduced if the magnetic beads are dried excessively.

5.10 The centrifuge tube is taken down from the magnetic rack and 40 μL of ultrapure water is added for vibration and uniform mixing.

5.11 Incubated at room temperature for 5 min for eluting DNA.

5.12 The mixture is centrifugalized transiently, the centrifuge tube is placed on the magnetic rack to clarify the liquid, and the library is transferred to a new centrifugal tube. The centrifugal tube is stored at 20° C. below zero.

6. Library Quality Inspection

2 μL DNA library is taken and concentration of the library is detected by using dsDNA HS Assay Kit.

III Library Hybridization Capturing

Library hybridization capturing is carried out by means of the detection panel and a self-produced kit, and the operating process is carried out according to specification of a product.

1. The library of total amount of 1p g is placed in the centrifugal tube and a blocking reagent is added according to the following table 8.

TABLE 8

| Reagent | Volume |
|---|---|
| Human Cot DNA | 5 μL |
| blocked oligonucleotide | 2 μL |
| DNA library | 1 μg |

2. The EP tube is sealed by the sealing film and is placed in a vacuum centrifugal concentration instrument to be evaporated (60° C., about 20 min to 1 hour). Pay attention to check whether the mixture is evaporated or not anytime.

3. DNA Degeneration and Hybridization

3.1 The hybridization solutions are added into evaporated 1.5 mL centrifugal tube and the system is configured according to the following table 9:

TABLE 9

| Reagent | Volume |
|---|---|
| hybridization buffer | 8.5 μL |
| Hybridization enhancer | 2.7 μL |
| Panel | 4 μL |
| Water without nuclease | 1.8 μL |

3.2 The mixture is vibrated and mixed uniformly, centrifugalized transiently and incubated at room temperature for 5 min.

3.3 The step 3.2 is repeated.

3.4 The liquid in the step 3.3 is transferred to a 200 μL PCR tube, the PCR tube is placed in the PCR instrument to be hybridized at 65° C. for 16 hours, and the hybridization process is as shown in table 10.

TABLE 10

| Temperature | time |
|---|---|
| 95° C. | 30 s |
| 65° C. | 4 h |
| 65° C. | Hold |

Lid temperature is 100° C.

4. Preparation of an Eluting Working Solution

4.1 A preparation method for capture buffer solution is as shown in table 11, and the buffer solution is prepared according to the table 11 according to the number of captures.

TABLE 11

| | | | l*Working |
|---|---|---|---|
| | | | solution |
| Reagent | Reagent/μL | Water/μL | volume/μL |
| Magnetic bead washing solution | 150 | 150 | 300 |
| Eluting working solution 1 | 25 | 225 | 250 |
| Eluting working solution 2 | 15 | 135 | 150 |
| Eluting working solution 3 | 15 | 135 | 150 |
| Eluting working solution 4 | 30 | 270 | 300 |

4.2 Sub-packaging of reagents needed to be incubated:

160 μL of eluting working solution 4 is sub-packaged into an 8-tube strip;

110 μL of eluting working solution 1 is sub-packaged into the 8-tube strip;

4.3 The magnetic beads, the eluting working solution 1 and the eluting working solution 4 are incubated and captured, and incubation is started when experiment is started, wherein the incubation time is about 45 min. The incubation flow is carried out according to a table 12.

TABLE 12

| Temperature | time |
|---|---|
| 65° C. | Hold |

Lid temperature is 70° C.

Room temperature balance is carried out for 30 min before the capture magnetic beads are used.

5. Purification after Hybridization:

5.1 Streptavidin magnetic beads

5.1.1 50 μL of Capture beads are sub-packaged in the 8-tube strip and 100 μL magnetic bead washing solution is added to vibrate and mix uniformly. The 8-tube strip is placed on the magnetic rack to clarify the liquid, and the supernatant is abandoned.

5.1.2 100 μL of magnetic bead washing solution is added to vibrate and mix uniformly. The 8-tube strip is placed on the magnetic rack to clarify the liquid, and the supernatant is abandoned.

5.1.3 100 μL of magnetic bead washing solution is added to vibrate and mix uniformly. The 8-tube strip is placed on the magnetic rack to clarify the liquid, and the supernatant is abandoned.

5.1.4 The 8-tube strip is taken down from the magnetic rack, the mixture is centrifugalized transiently, and is placed on the magnetic rack, and residual liquid at the bottom of the tube is abandoned thoroughly by using a 10 μL pipette tip.

5.1.5 The magnetic bead re-suspending mixed solution is added into the cleaned magnetic beads, and the system is configured as shown in table 13.

TABLE 13

| Reagent | Volume |
|---|---|
| Hybridization buffer solution | 8.5 μL |
| Hybridization enhancer | 2.7 μL |
| Water without nuclease | 5.8 μL |

5.1.6 The mixture is vibrated and mixed uniformly fully, centrifugalized transiently, transferred to the PCR tube and placed in the PCR instrument at 65° C. (lid temperature 70° C.) to be incubated for 15 min.

5.2 The hybridization solution captured overnight is measured by a pipettor to ensure that the volume of the hybridization solution captured overnight is 17 μL to prevent from the loss.

5.3 The incubated magnetic bead re-suspending mixed solution with the magnetic beads at 65° C. is transferred to the hybridization solution captured overnight, and the hybridization solution is blowed and mixed uniformly by using a pipettor (in the whole incubation process, the PCR tube must not to be separated from 65° C., and in all uniform mixing steps, blowing and uniform mixing on the 65° C. PCR instrument are carried out by using the pipettor). The solution is placed in the PCR instrument to be incubated for 45 min at 65° C. (PCR lid temperature is set at 70° C.), and blown and mixed uniformly by a pipettor once at an interval to ensure that the magnetic beads are suspended. The time intervals are 11 min, 11 min, 11 min and 12 min.

5.4 Hot washing (important: in the whole hot washing process, the temperature is not lower than 65° C. to the greatest extent):

5.4.1 After incubation, 100 μL of 65° C. preheated eluting working solution 1 is added into the 8-tube strip, and blown and mixed uniformly by using the pipettor. The 8-tube strip is placed on the magnetic rack to clarify the liquid, and the supernatant is abandoned.

5.4.2 The 8-tube strip is taken down from the magnetic rack, the mixture is centrifugalized transiently, and is placed on the magnetic rack, and residual liquid at the bottom of the tube is abandoned thoroughly by using a 10 μL pipette tip.

5.4.3 150 μL of 65° C. preheated eluting working solution 14 is added, the solution is blown and mixed uniformly by using the pipettor, is incubated for 5 min at 65° C., and is placed on the magnetic rack for 1 min to clarify the liquid, and the supernatant is abandoned.

5.4.4 150 μL of 65° C. preheated eluting working solution 14 is added, the solution is blown and mixed uniformly by using the pipettor, is incubated for 5 min at 65° C., and is placed on the magnetic rack for 1 min to clarify the liquid, and the supernatant is abandoned.

5.4.5 The 8-tube strip is taken down from the magnetic rack, the mixture is centrifugalized transiently, and is placed on the magnetic rack, and residual liquid at the bottom of the tube is abandoned thoroughly by using a 10 μL pipette tip.

5.5 Normal temperature washing

5.5.1 150 μL of 65° C. preheated eluting working solution 1 is added, vibrated for 30 s, stand for 30 s, then vibrated for 30 s and stand for 30 s (totally 2 min), the solution is centrifugalized transiently, and is placed on the magnetic rack for 1 min to clarify the liquid, and the supernatant is abandoned. The 8-tube strip is taken down from the magnetic rack, the mixture is centrifugalized transiently, and is placed on the magnetic rack, and residual liquid at the bottom of the tube is abandoned thoroughly by using a 10 μL pipette tip.

5.5.2 150 μL of 65° C. preheated eluting working solution 2 is added, vibrated for 30 s, stand for 30 s, then vibrated for 30 s and stand for 30 s (totally 2 min), the solution is centrifugalized transiently, and is placed on the magnetic rack for 1 min to clarify the liquid, and the supernatant is abandoned. The 8-tube strip is taken down from the magnetic rack, the mixture is centrifugalized transiently, and is placed on the magnetic rack, and residual liquid at the bottom of the tube is abandoned thoroughly by using a 10 μL pipette tip.

5.5.3 150 μL of the eluting working solution 3 placed at room temperature is added, vibrated for 30 s, stand for 30 s, then vibrated for 30 s and stand for 30 s (totally 2 min), the solution is centrifugalized transiently, and is placed on the magnetic rack for 1 min to clarify the liquid, and the supernatant is abandoned. The 8-tube strip is taken down from the magnetic rack, the mixture is centrifugalized transiently, and is placed on the magnetic rack, and residual liquid at the bottom of the tube is abandoned thoroughly by using a 10 μL pipette tip.

5.5.4 20 μL ultrapure water is added into the centrifugal tube for vibration and uniform mixing for next amplification test.

6. PCR after Capturing

6.1 Adding to the reaction system according to the following table 14:

TABLE 14

| Reagent | Volume |
|---|---|
| Hot start enzyme | 25 μL |
| Primer, 5 μM | 5 μL |
| DNA eluted in the previous step | 20 μL |

6.2 DNA is mixed uniformly by vortex, centrifugalized transiently and placed in the PCR instrument, and PCR reaction is carried out as shown in the following table 15.

TABLE 15

| Step | Temperature | time | Cycle number |
|---|---|---|---|
| Predegeneration | 98° C. | 45 sec | 1 |
| Degeneration | 98° C. | 15 sec | |
| Annealing | 60° C. | 30 sec | 14 |
| Extending | 72° C. | 30 sec | |
| Re-extending | 72° C. | 1 min | 1 |
| Save | 8° C. | ∞ | 1 |

7. Purification after Amplification

7.1 The amplified captured DNA library is placed on the 96 porous magnetic plate to detect concentration, and it is ensured that the previous experiment is accurate.

7.2 The purified magnetic beads are taken out and balanced at room temperature for 30 min for later use.

7.3 75 μL of purified magnetic beads are placed in a 1.5 mL low adsorption centrifugal tube, 50 μL of the amplified capture DNA library supernatant is added to be vibrated and mixed uniformly, and incubated at room temperature for 10 min.

7.4. The 8-tube strip is placed on the magnetic rack for 1 min to clarify the liquid, and the supernatant is abandoned.

7.5. The 1.5 mL low adsorption centrifugal tube is taken down from the magnetic rack, the mixture is centrifugalized transiently, and is placed on the magnetic rack, and residual liquid at the bottom of the tube is abandoned thoroughly by using a 10 μL pipette tip.

7.6 200 μL of 80% ethanol is added to incubate for 30 see to be abandoned. Note: 80% ethanol is prepared when it is in need. The cleaning step by 200 μL of 80% ethanol is repeated once.

7.7. The 1.5 mL low adsorption centrifugal tube is taken down from the magnetic rack, the mixture is centrifugalized transiently, and is placed on the magnetic rack, and residual liquid at the bottom of the tube is abandoned thoroughly by using a 10 μL pipette tip. The solution is dried at room temperature till ethanol is volatilized fully (the magnetic beads do not reflective from the front surface and the back surface is dried). Note: the DNA output will be reduced if the magnetic beads are dried excessively.

7.8 The centrifuge tube is taken down from the magnetic rack and 40 μL of ultrapure water is added for vibration and uniform mixing. Incubated at room temperature for 2 min.

7.9 The mixture is centrifugalized transiently, the centrifuge tube is placed on the magnetic rack for 1 min to clarify the liquid, and the captured sample is transferred to a new centrifugal tube.

8. Quality Inspection:

2 μL capture sample is taken to detect Qubit concentration.

(II) Breaking, Library Constructing and Capturing Steps for a Blood Cell Sample

Blood cells are extracted by employing a Tiangen extraction kit and the operating process is conducted according to a product specification. The extracted DNA is quantified by using Qubit 3.0 and dsDNA HS Assay Kit.

I Library Construction

1. Blood Cell DNA Fragmentation/End Repair/a Addition

1.1 200 ng of blood cell DNA sample is taken according to the qubit quantitative result and is diluted to 17.5 μL by using H₂O. The reaction system is prepared according to the following table 16:

TABLE 16

| Component name | Volume |
|---|---|
| W*FEA buffer solution | 2.5 μL |
| DNA sample | 17.5 μL ( 200 ng) |
| 5*FEA enzyme mixed solution | 5 μL |
| Total volume | 25 μL |

1.2 DNA is mixed uniformly by vortex, micro-centrifugalized and placed in a PCR instrument, and the reaction program is as shown in the following table 17:

TABLE 17

| Reaction step | Reaction temperature | Reaction time |
|---|---|---|
| 1 | 4° C. | 1 min |
| 2 | 32° C. | 20 min |
| 3 | 65° C. | 30 min |
| 4 | 4° C. | ∞ |

Adapter Connection

2.1 Adapter preparation: 2.5 μL of adapter is added with 2.5 μL water to be diluted to 5 μL.

2.2 Corresponding reagents are added into a reaction tube according to the following table 18:

TABLE 18

| Component name | Volume ( μL) |
|---|---|
| Reaction product | 25 |
| Ligase buffer solution | 10 |
| DNA ligase | 5 |
| Water without nuclease | 5 |
| Total volume | 45 |

2.3 The mixture is mixed uniformly by vortex, is micro-centrifugalized and is placed in a PCR instrument to be incubated for 30 min at 20° C.

3. Purification after Connection:

3.1 Beckman Agencourt AMPure XP magnetic beads are sub-packaged to a new 8-tube strip, 40 μL (0.8×) in each. Note: the magnetic beads are placed for 30 min at room temperature in advance before use.

3.2 After PCR in the previous step is finished, the sample is taken out, centrifugalized transiently and transferred to the sub-packaged 40 μL magnetic bead centrifugal tube, i.e. a system in the following table 19:

TABLE 19

| Reagent | Volume |
|---|---|
| Connecting product | 50 μL |
| Magnetic beads | 40 μL ( 0.8×) |
| Total volume | 90 μL |

3.3 The mixture is vibrated and mixed uniformly, and incubated for 10 min at room temperature, so that DNA and the magnetic beads are combined fully. Note: a pipe cover is pressed during vibration. The mixture is centrifugalized transiently, the centrifuge tube is placed on the magnetic rack to clarify the liquid, and the supernatant is abandoned. Note: do not absorb the magnetic beads.

3.4 200 μL of 80% ethanol is added to incubate for 30 see to be abandoned. The washing step by 200 μL of 80% ethanol is repeated once. Note: 80% ethanol is prepared when it is in need.

3.5 Residual ethanol at the bottom of the centrifugal tube is absorbed thoroughly with a 10 μL pipette tip and is dried for 3-5 min at room temperature till ethanol is volatilized fully. Note: the DNA output will be reduced if the magnetic beads are dried excessively.

3.6 The centrifuge tube is taken down from the magnetic rack and 13 μL of ultrapure water is added for vibration and uniform mixing. Note: the pipe cover is pressed during vibration. incubated at room temperature for 5 min for eluting DNA.

3.7 The mixture is centrifugalized transiently, the centrifuge tube is placed on the magnetic rack to clarify the liquid, and 10 μL of supernatant is transferred to a new PCR pipe for a next amplification test.

4. Library Amplification

4.1 Adding to the reaction system according to the following table 20:

TABLE 20

| Reagent components | Volume |
|---|---|
| Hot start enzyme | 12.5 μL |
| Mixture of primer and reaction buffer solution | 2.5 μL |
| Adapter connecting library | 10 μL |
| Total volume | 25 μL |

4.2 The mixture is mixed uniformly by vortex, is micro-centrifugalized and is placed in a PCR instrument, and the reaction program is as shown in the following table 21:

TABLE 21

| Step | Temperature | time | Cycle number |
|---|---|---|---|
| Predegeneration | 98° C. | 45 sec | 1 |
| Degeneration | 98° C. | 15 sec | 6 |
| Annealing | 60° C. | 30 sec | |
| Extending | 72° C. | 30 sec | |
| Final extending | 72° C. | 1 min | 1 |
| Save | 4° C. | ∞ | 1 |

5. Acquisition of DNA

5.1 Beckman Agencourt AMPure XP magnetic beads are sub-packaged to new 8-tube strips, 17.5 μL and 7.5 μL, respectively.

5.2 After PCR in the previous step is finished, the sample is taken out.

5.3 The mixture is centrifugalized transiently and is transferred to 17.5 μL of sub-packaged Beckman Agencourt AMPure XP magnetic beads. That is, the reaction system is as shown in the following table 22:

TABLE 22

| Reagent | Volume |
|---|---|
| PCR product | 25 μL |
| Magnetic beads | 17.5 μL |
| | (0.7 ×) |
| Total volume | 42.5 μL |

5.4 The mixture is vibrated and mixed uniformly, and is incubated for 15 min at room temperature, so that DNA and the magnetic beads are combined fully. Note: a pipe cover is pressed during vibration.

5.5 The mixture is centrifugalized transiently, the centrifuge tube is placed on the magnetic rack.

5.6 The supernatant is transferred to 7.5 μL of Beckman Agencourt AMPure XP magnetic beads after being clarified. Note: do not absorb the magnetic beads.

5.7 The mixture is vibrated and mixed uniformly, and incubated for 10 min at room temperature, so that DNA and the magnetic beads are combined fully. Note: the pipe cover is pressed during vibration.

5.8 The mixture is centrifugalized transiently, the centrifuge tube is placed on the magnetic rack to clarify the liquid, and the supernatant is abandoned. Note: do not absorb the magnetic beads.

5.9 200 μL of 80% ethanol is added to incubate for 30 see to be abandoned. Note: 80% ethanol is prepared when it is in need. The washing step by 200 μL of 80% ethanol is repeated once.

5.10 Residual ethanol at the bottom of the centrifugal tube is absorbed thoroughly with a 10 μL pipette tip and is dried for 3-5 min at room temperature till ethanol is volatilized fully. Note: the DNA output will be reduced if the magnetic beads are dried excessively.

5.11 The centrifuge tube is taken down from the magnetic rack and 70 μL of ultrapure water is added for vibration and uniform mixing.

5.12 Incubated at room temperature for 5 min for eluting DNA.

5.13 The mixture is centrifugalized transiently, the centrifuge tube is placed on the magnetic rack to clarify the liquid, and the library is transferred to a new centrifugal tube.

6. Library Quality Inspection:

2 μL of DNA library is taken to detect concentration detection.

II Library Hybridization Capturing

Hybridization capture is the same as the library hybridization capture step of library construction and capture of tissue DNA.

III. Detection Result

The detection result of point mutation, variation of copy number and rearrangement of the standard substance is as shown in table 23. By taking the ddPCR detection result as a golden standard, a kit of the company is excellent in detection performance on point mutation, rearrangement and variation of copy number of the tissue sample.

TABLE 23

| | | NGS | | | | | | |
|---|---|---|---|---|---|---|---|---|
| Sample type | Mutation type | Cutoff | Sensitivity | | Specificity | PPV | LOD95 | Accuracy |
| Cell line DNA | Point mutation | 0.3% | 96.55% | (MAF > 1%) | 100% | 100% | 1% | 96.0% |
| analog tissue | Variation of copy | 2.2 | 100% | (CNV > 2.5) | 100% | 100% | 2.4 | |
| (50 ng) | numbers | | | | | | | |
| | Rearrangement | 2 reads | 100% | (MAF > 0.3%) | 100% | 100% | NA | |
| Cell line DNA | Point mutation | 0.3% | 98.85% | (MAF > 1%) | 100% | 100% | 0.1% | |
| analog tissue | Variation of copy | 2.2 | 100% | (CNV > 2.5) | 100% | 100% | 2.4 | |
| (200 ng) | numbers | | | | | | | |
| | Rearrangement | 2 reads | 100% | (MAF > 0.3%) | 100% | 100% | NA | |

(III) Detection Method for Co-Deletion of Chromosomes 1p/19q

1. Processing of Fastq Data as an Input File Usable by Software

a) Alignment

bwa-0.7.12 mem is called to make paired reads alignment on each pair of fastq files to a hg19 human reference genomics sequence. Besides, −M parameter and ID with appointed Reads Group, other parameter options are not used, and an initial bam file is generated;

b) Sequencing

The SortSam module of Picard-2.1.0 is called to sequence the initial bam file according to the chromosomal location, and the parameter is set as “ORT_ORDER-coordinate”;

c) Selecting

SAMtools-1.3view is called to select the sequenced bam file and “−F0x900” is taken as the parameter;

D) MarkDuplicates

The MarkDuplicates module of Picard-2.1.0 is called to mark repeated sequences in the selected bam file. During follow-up analysis, the repeated sequences will be filtered, and data without duplications is configured to analysis;

e) Index Establishment

The index module of SAMtools-1.3 is called to establish an index for the finally generated bam file and generate a bai file paired with the bam file with marked duplications;

f) SNP Detection

The mpileup module of SAMtools is used first to generate a mileup file according to the bam file and a bed file of each sample and a fasta file of the human reference genomic sequence, and a mutation list vcf file of each sample is generated according to the mpileup file by using a mpileup2cns module of VarScan.

2. Co-Deletion Method for 1p and 19q Based on SNP with a Control Sample

During identification, SNP detection result files of the control sample and the to-be-tested sample are taken as input files, and the system of the present disclosure is configured to select the files.

One sample is selected for FISH 1p/19q detection and method of the embodiment simultaneously. A result is as shown in FIGS. 3 and 4. The FISH and NGS detection results show 1p/19q loss. It is illustrated that the embodiment is consistent with the FISH detection result.

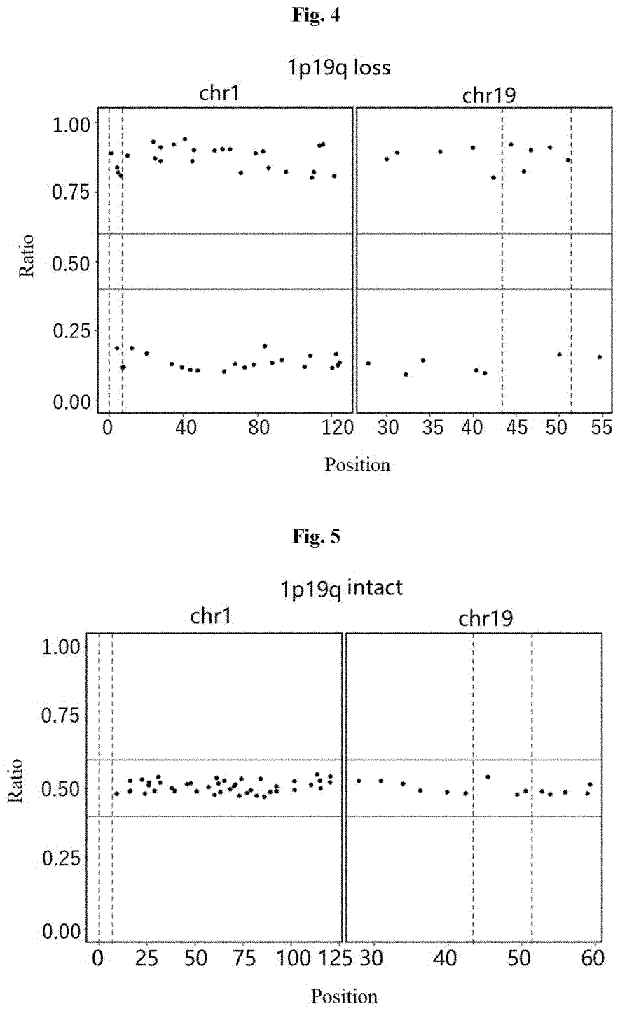

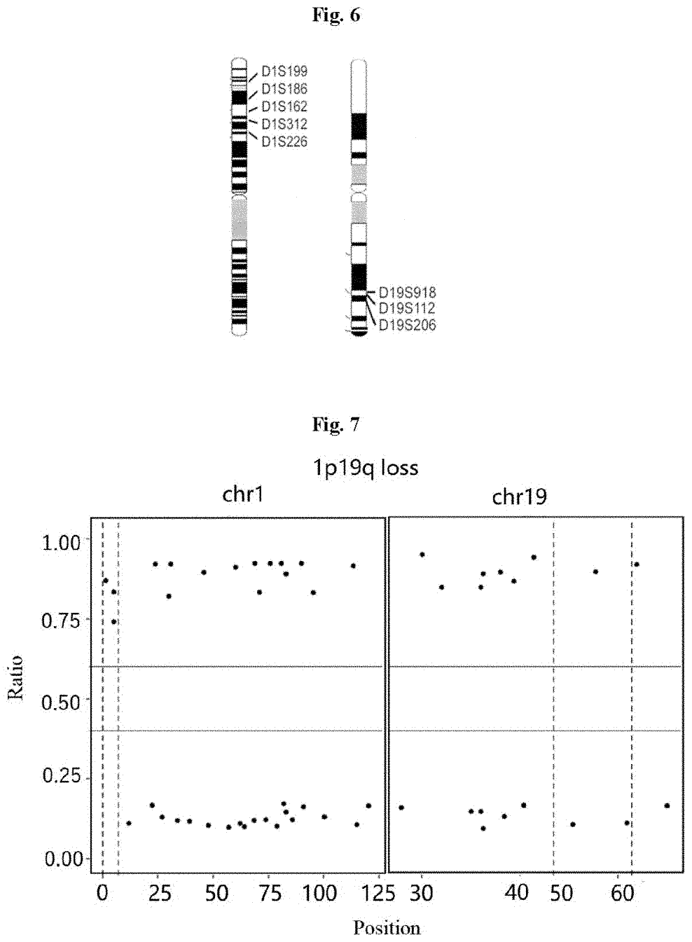

One sample is selected for method detection of the embodiment and one generation sequencing detection simultaneously. A result is as shown in FIGS. 5 and 6. One generation sequencing result is positive and NGS detection (the method of the embodiment) result is co-deletion negative. As the sample IDH is wild, 1p19q shall be negative, so that the result shows that NGS detection is more accurate than one generation detection.

3. Co-Deletion Method for 1p and 19q Based on SNP without a Control Sample

a) Control Set Establishment

A group of 60 control samples are used, the SNP detection result files of the 60 samples are taken as input file, and the system of the present disclosure is used to establish a control set file.

b) Co-Deletion Identification for 1p and 19q

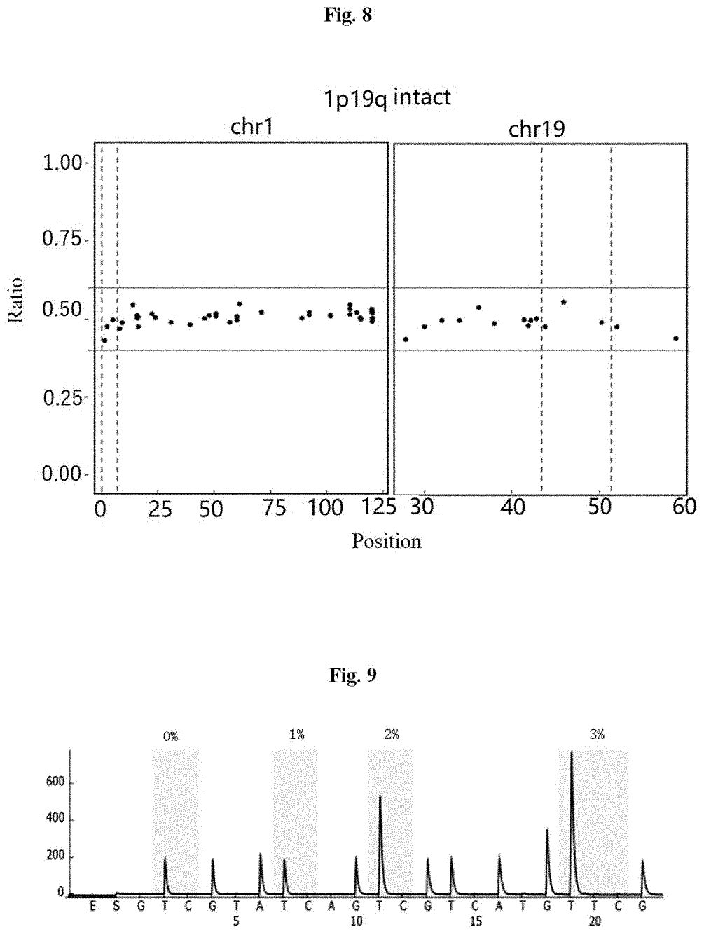

An SNP detection result file of the to-be-tested sample and the control set are taken as input files of the embodiment, three types of the same two samples are identified by using the present disclosure, a result is as shown in FIGS. 7 and 8, and the judgment is accurate.

4. Co-Deletion Method for 1p and 19q Based on SNP with Control Sample

During identification, alignment result files of the control sample and the to-be-tested sample are taken as input files, and the system of the present disclosure is used to identify three types of two samples, and each STR identification result is as shown in the following table 24:

TABLE 24

| Chrom | Loci | R(LOH) | | R(NoLOH) | |
|---|---|---|---|---|---|
| 1 p | STR1p_1 | 0.243 | Abnormal | 0.799 | Normal |
| | STR1p_2 | 0.176 | Abnormal | 0.851 | Normal |
| | STR1p_3 | / | Hom | 0.784 | Normal |
| | STR1p_4 | 0.221 | Abnormal | / | Hom |
| | STR1p_5 | 0.279 | Abnormal | 0.760 | Normal |
| | STR1p_6 | 0.387 | Abnormal | / | Hom |
| | STR1p_7 | 0.408 | Abnormal | 0.864 | Normal |
| | STR1p_8 | / | Hom | 0.796 | Normal |
| | STR1p_9 | 0.318 | Abnormal | / | Hom |
| | STR1p_10 | 0.431 | Abnormal | 0.737 | Normal |
| | STR1p_11 | 0.253 | Abnormal | 0.766 | Normal |
| | STR1p_12 | / | Hom | 0.865 | Normal |
| | STR1p_13 | / | Hom | / | Hom |
| | STR1p_14 | 0.268 | Abnormal | 0.757 | Normal |
| | STR1p_15 | 0.194 | Abnormal | 0.835 | Normal |
| | STR1p_16 | 0.315 | Abnormal | / | Hom |
| | STR1p_17 | 0.199 | Abnormal | 0.655 | Normal |
| | STR1p_18 | 0.257 | Abnormal | 0.806 | Normal |
| 19q | STR19q_1 | 0.343 | Abnormal | 0.684 | Normal |
| | STR19q_2 | / | Hom | 0.856 | Normal |
| | STR19q_3 | 0.232 | Abnormal | / | Hom |
| | STR19q_4 | 0.231 | Abnormal | / | Hom |
| | STR19q_5 | 0.321 | Abnormal | 0.857 | Normal |
| | STR19q_6 | / | Hom | 0.790 | Normal |
| | STR19q_7 | 0.315 | Abnormal | 0.511 | Normal |
| | STR19q_8 | 0.277 | Abnormal | 0.627 | Normal |

Final result is settled in the following table 25:

TABLE 25

| | LOH | NOLOH |
|---|---|---|
| 1 p | 14/14 | 0/13 |
| 19q | 6/6 | 0/6 |
| Final | Abnormal | Normal |

5. CNV-Based Co-Deletion Method for 1p and 19q

a) Cnv Base Line Establishment

30 blood cell samples without abnormal conditions in copy number are selected as reference crowd group samples and a same mode is adopted for capturing sequencing and pre-processing of sequencing data. The bam files of the 30 samples, the bed file recording the capture region and the fastq file of the human reference genomic sequence as input files, and the system of the disclosure is used to generate COV and GCS files of the reference crowd group.

b) CNV Detection

The bam file of the to-be-tested sample and the COV and GCS files of the reference crowd group are input to identify the copy numbers of genes covered in capture region of each sample to obtain RZ, COV and GCS files of each file and final two SCNA result files.

c) 1p/19q Detection Result is as Shown in Table 26.

TABLE 26

| | 1 p | 19q |
|---|---|---|
| LOH | 1.336 | 1.291 |
| | (loss) | (loss) |
| NOLOH | 1.82 | 2.057 |
| | (normal) | (normal) |

According to the copy number in table 26, it can be seen from the result that loss occurs on 1p and 19q of the LOH sample simultaneously and 1p and 19q of the non-LOH sample are neutral. It is illustrated that the detection result of the next generation sequencing-based system for detecting co-deletion of 1p/19q of glioma is accurate.

(VI) Detection Method for MGMT Promoter Methylation

1. Extracting genomic DNA of the to-be-tested sample

2. Converting the genomic DNA by bisulfite

2.1 The starting amount of the converted DNA is 100 ng, the initial volume of the sample is 20 μL, and if the volume of the sample is not smaller than 20 μL, the volume is supplemented by water.

2.2 130 μL of a bisulfite conversion reagent is added into the DNA sample, the mixture is vibrated and mixed uniformly, is centrifugalized transiently and is placed on the PCR instrument for PCR reaction according to the following table 27:

TABLE 27

| Temperature | time |
|---|---|
| 98° C. | 8 min |
| 54° C. | 60 min |
| 4° C. | 20 h |

2.3 600 μL of M-combination solution is added into a filter column, the product after the reaction in the step 2.2 is added into the filter column containing the M-combination solution, and the mixture is blown and mixed uniformly, and stand for 2 min, and is centrifugalized for 1 min at 12000 rpm.

2.4 The liquid in a collecting tube is added again into an adsorption column to stand for 2 min and is centrifugalized for 1 min at 12000 rpm, and waste liquid is abandoned.

2.5 100 μL of M-washing solution is added, is centrifugalized for 1 min at 12000 rpm, and waste liquid is abandoned.

2.6 200 μL of L-desulfonation reagent to be incubated for 15-20 min at room temperature (20-30° C.), and after incubation, the mixture is centrifugalized for 1 min at 12000 rpm, and waste liquid is abandoned.

2.7 200 μL of M-washing solution is added, is centrifugalized for 1 min at 12000 rpm, and waste liquid is abandoned.

2.8 The step 1.8 is repeated, 200 μL M-washing solution is added, is centrifugalized for 1 min at 12000 rpm, and waste liquid is abandoned.

2.9 The adsorption column is placed back in the collecting tube, is centrifugalized for 1 min at 12000 rpm and the waste liquid is poured. The adsorption column is uncapped and is placed at room temperature for 2-5 min to air residual rinsing solution in an adsorption material.

2.10 The adsorption column is transferred to a clean centrifugal tube, 20 μL of an eluting buffer solution TE which is preheated at 50° C. is dropwise added into a middle portion of an adsorption film to elute, and the clean centrifugal tube is placed at room temperature for 2-5 min, and is centrifugalized for 1 min at 12000 rpm.

2.11 The liquid in the collecting tube is added again into the adsorption column to stand for 5 min at room temperature and is centrifugalized for 1 min at 12000 rpm, and the centrifugal tube collecting the converted DNA is stored at 20° C. below zero.

3. MGMT gene amplification

3.1 A mix is prepared according to the following table 28 and is vibrated and mixed uniformly.

TABLE 28

| Reagent | Volume |
|---|---|
| Hot start U enzyme | 12.5 μL |
| Primer MGMT F | 1 μL |
| Primer MGMT R | 1 μL |
| Converted DNA | 5 μL |
| Water | 5.5 μL |
| Total volume | 25 μL |

The primers for detecting the MGMT promoter methylation include a pair of specific amplification primers, the primer sequences being as shown in the following table 29.

TABLE 29

| Name | Sequences (5′-3′) |
|---|---|
| MGMT F (SEQ ID NO: 1) | tygygttttggatatgttgg |
| MGMT F (SEQ ID NO: 2) | craaaaaaaactccrcactc |

3.2 The converted DNA in the previous step is added into a mixed solution of table 25 to be vibrated and mixed uniformly.

3.3 The mixture is centrifugalized transiently and is placed on the PCR instrument for PCR reaction as shown in the following table 30.

TABLE 30

| Temperature | time | Cycle number |
|---|---|---|
| 95° C. | 2 min | 1 |
| 98° C. | 30 sec | 35 |
| 50° C. | 30 sec | |
| 72° C. | 1 min | |
| 72° C. | 10 min | 1 |
| 4° C. | hold | 1 |

4. Beckman Agencourt AMPure XP magnetic bead purification Library construction and sequencing for the PCR product are carried out by way of DNA NGS library construction.

V. Detection Result

10 samples are subjected to pyrosequencing and NGS MGMT detection simultaneously, and a result is shown in table 31. The detection results of the 10 samples are consistent.

TABLE 31

| Sample number | | | | | | | | | | |
|---|---|---|---|---|---|---|---|---|---|---|
| The detection panel | Negative | Positive | Negative | Positive | Positive | Negative | Positive | Negative | Weakly | Negative |
| of the present | | | | | | | | | positive | |
| disclosure | | | | | | | | | | |
| Pyrosequencing | Negative | Positive | Negative | Positive | Positive | Negative | Positive | Negative | Weakly | Negative |
| | | | | | | | | | positive | |

### Embodiment 2

Detection of Amplification Result on Annealing Temperature, Working Concentration and PCR Cycle Numbers of MGMT Gene Promoter Methylation Primer Reagents and makers needed in the embodiment are as shown in table 32:

TABLE 32

| Reagent | maker |
|---|---|
| KAPA HiFi HS Uracil + RM | KAPA |
| KAPA Hyper Prep kit | KAPA |
| EZ DNA Methylation-lightning Kit | EZ |

I. Extraction of clinical sample tissue DNA

II Steps of converting genomic DNA by bisulfite, performing MGMT amplification and the like are referred to the embodiment 1.

III Selection of different primer annealing temperatures, working concentrations and PCR cycle numbers

3.1 Selection of annealing temperatures of the primers: 40° C., 45° C., 50° C., 55° C., 60° C.

3.2 Selection of working concentrations of the primers: 4 μM, 5 μM, 10M, 15 M, 16 μM.

3.3 Selection of PCR cycle numbers: 25 cycles, 30 cycles, 35 cycles, 40 cycles and 45 cycles.

IV. Detection result:

4.1 The detection result of the annealing temperatures of the primers is as shown in table 33:

TABLE 33

| Annealing | |
|---|---|
| temperature | Test result |
| 40° C. | More nonspecific amplification |
| 45° C. | The correct targeted stripe is amplified |
| 50° C. | The correct targeted stripe is amplified |
| 55° C. | The correct targeted stripe is amplified |
| 60° C. | No amplified stripe |

4.2 The detection result of the working concentrations of the primers is as shown in table 34:

TABLE 34

| Working | |
|---|---|
| concentration | Test result |
| 4 μM | No amplified stripe |
| 5 μM | The correct targeted stripe is amplified |
| 10 μM | The correct targeted stripe is amplified |
| 15 μM | The correct targeted stripe is amplified |
| 16 μM | More dimers of the primers |

4.3 The detection result of PCR cycle numbers is as shown in table 35

TABLE 35

| PCR cycle | |
|---|---|
| number | Test result |
| 25 cycles | No amplified stripe |
| 30 cycles | A correct targeted stripe is amplified |
| 35 cycles | A correct targeted stripe is amplified |
| 40 cycles | A correct targeted stripe is amplified |
| 45 cycles | An over-amplified phenomenon occurs, |
| | which may increase the polluting risk. |

### Embodiment 3

Processing Method for Sequencing Data of MGMT Gene Methylation

1) Alignment

bismark is called to align each pair of fastq files as paired reads to the MGMT human reference genomic sequence, an initial bam file is generated and the parameter is set as “--phred33-quals”.

II) Sequencing

The sort module of SAM tools is called to sequence the initial bam file according to a chromosomal location, and the parameter is default.

III Adding Read Group information

The Add Or Replace Read Groups module of Picard is called to add Read Group information to the sequence bam file, and the parameter is set as “VALIDATION STRINGENCY=LENIENT”.

IV Removing Overlapped Interval Between Double End Sequences

The clip Overlap module of Bam Util is called to remove overlapped sequences between the double end sequences in the aligned bam file, and the overlapped sequences are not filtered in follow-up analysis, such that calculation of Beta value will be affected.

V) Index Establishment

The index module of SAMtools is called to establish an index for the finally generated bam file and generate a bai file paired with the bam file without duplications.

VI Data Correction

The Bisulfite Count Covariates module and Bisulfite Table Recalibration module of BisSNP are used successively to correct the processed bam file, bed file (a file input manually, wherein the file records location information of the human reference genomic sequence), the fasta file of the human reference genomic sequence and the vcf file emerging frequently among human to remove low quality (including sequencing quality and/or aligning quality) loci, thereby improving the identifying accuracy.

VII SNP/Methylation Locus Joint Identification

SNP/methylation loci are identified simultaneously by using a Bisulfite Genotyper module to respectively obtain initial vcf files of SNP (non-focused loci, the data can be in not use) and methylation (i.e. CpG loci).

VIII Sequencing of Methylated Loci

The sort By Ref And Cor module of BisSNP is used to arrange the initially identified methylated vcf files according to genomic locations.

VIIII Filtration of Methylated Loci

The VCF post process of BisSNP is used to filter the methylated vcf files sequenced subsequently.

X. Data Arrangement

The filtered methylated vcf files are arranged to readable file formats to obtain methylated detection results, specifically as shown in table 36.

TABLE 36

| | | | | | | | Number of |
|---|---|---|---|---|---|---|---|
| | | | | | | | reads |
| | | | | | Number of | | covering |
| | | | forward | Methylation | reads | Methylation | the locus |
| | | | and | level of | covering the | level of | by the |
| | Initiation | Termination | reverse | first | locus by the | second | second |
| Chromosome | locus | locus | strands | sample | first sample | sample | sample |
| chr10 | 131265494 | 131265495 | + | 0.86 | 5500 | 0 | 10641 |
| chr10 | 131265496 | 131265497 | + | 0.88 | 5498 | 0 | 10602 |
| chr10 | 131265507 | 131265508 | + | 0.87 | 5497 | 0.01 | 10646 |
| chr10 | 131265514 | 131265515 | + | 0.88 | 5499 | 0.01 | 10646 |
| chr10 | 131265519 | 131265520 | + | 0.86 | 5500 | 0.01 | 10647 |
| chr10 | 131265522 | 131265523 | + | 0.84 | 5500 | 0 | 10646 |
| chr10 | 131265526 | 131265527 | + | 0.43 | 5498 | 0 | 10647 |
| chr10 | 131265536 | 131265537 | + | 0.25 | 5499 | 0 | 10647 |
| chr10 | 131265538 | 131265539 | + | 0.89 | 5498 | 0.01 | 10644 |
| chr10 | 131265543 | 131265544 | + | 0.89 | 5500 | 0.01 | 10646 |
| chr10 | 131265548 | 131265549 | + | 0.87 | 5500 | 0.01 | 10646 |
| chr10 | 131265554 | 131265555 | + | 0.89 | 5499 | 0.01 | 10644 |

| | | |
|---|---|---|
| MGMTNGS result | 59.5% Positive | 0.25% Negative |
| Pyrophosphoric acid result | 36% Positive | 2.25% Negative |

Appendix: Judging standard for positiveness in the above table is positive when the methylation level is over 10%.

### Embodiment 4

Repeatability Estimation of MGMT Gene Methylation Detection

I Sample Preparation

3 batches of MGMT standard substances with same mutation frequency (theoretical mutation frequencies are 10.00%, 15% and 20% respectively) are prepared, repeatability detection on the 3 batches of samples is carried out, and methylation frequency of detection of the 3 batches of samples is counted.

II Amplification of the targeted region and construction of amplicon library for sequencing detection, specifically, steps of converting the genomic DNA by bisulfite and performing MGMT amplification and the like are referred to the embodiment 1, and an analytical flow of sequencing data is referred to the embodiment 3.

III Detection result: the methylation frequency of 3 batches of detection is as shown in table 37.

TABLE 37

| Methylation frequency | | | | Standard | |
|---|---|---|---|---|---|
| Batch 1 | Batch 2 | Batch 3 | mean | deviation | CV |
| 8.00% | 8.00% | 8.00% | 8.00% | 0% | 0% |
| 13.00% | 13.50% | 12.00% | 12.83% | 0.76% | 5.95% |
| 18.50% | 16.50% | 17.25% | 17.42% | 1.01% | 5.80% |

It can be seen from the table 37 that CV (Coefficient of Variation) among the 3 batches in the detection results are small and good in repeatability.

### Embodiment 5

Consistence of MGMT Gene Methylation Detection and Pyrophosphoric Acid Detection for Clinical Sample

I. Extraction of clinical sample tissue DNA

II Steps of converting the genomic DNA by bisulfite and performing MGMT amplification and the like are referred to the embodiment 1, and an analytical flow of sequencing data is referred to the embodiment 3. Pyrosequencing is adopted simultaneously for verification and aligning and contrasting.

III The methylation level detection and judging result of the clinical sample is as shown in table 38.

TABLE 38

| | Methylation level | | Methylation level | |
|---|---|---|---|---|
| Sample | and detection result | | and detection result | |
| number | of pyrosequencing | | of MGMT NGS | |
| 1 | 2.75% | Negative | 0.75% | Negative |
| 2 | 36% | Positive | 59.5% | Positive |
| 3 | 1.75% | Negative | 1% | Negative |
| 4 | 11.5% | Positive | 18.25% | Positive |
| 5 | 42.25% | Positive | 50.75% | Positive |
| 6 | 1.75% | Negative | 1% | Negative |
| 7 | 28.5% | Positive | 39% | Positive |
| 8 | 1.75% | Negative | 1% | Negative |
| 9 | 8.5% | Negative | 4.75% | Negative |
| 10 | 2.25% | Negative | 0.25% | Negative |

Note:
it should be noted that the methylation level of each sample is measured according to the average methylation levels of four loci detected by pyrosequencing, and the sample with the methylation level which reaches 10% or above is judged as positive.

It can be seen from table 38 that the clinical sample is detected. Compared with the pyrosequencing detection method, the verification result shows that the MGMT NGS detection result of the present disclosure is consistent with that of pyrosequencing, indicating that an amplicon obtained by amplifying the primer obtains the methylation state of the MGMT gene promoter through high throughput sequencing and improved methylation analytical flow and the accuracy is not reduced to increase of the sequencing throughput.

### Embodiment 6

Advantage of MGMT Gene Methylation Detection Against Pyrosequencing

I. The methylated loci detected by two different methods are counted, and the counting result is as shown in table 39.

TABLE 39
MGMT NGS detection loci
and pyrosequencing detection loci

| | | Pyrosequencing |
|---|---|---|
| MGMT NGS | | detection |
| detection loci | | loci |
| 131265494 | 131265526 | 131265519 |
| 131265496 | 131265536 | 131265522 |
| 131265507 | 131265538 | 131265526 |
| 131265514 | 131265543 | 131265536 |
| 131265519 | 131265548 | / |
| 131265522 | 131265554 | / |

It can be seen from table 39 that the number of the methylated loci detected by the amplification library constructed by the primer of the present disclosure and the improved analytical flow for sequencing data is remarkably greater than that of the loci capable of being detected by an existing pyrophosphate detection method.

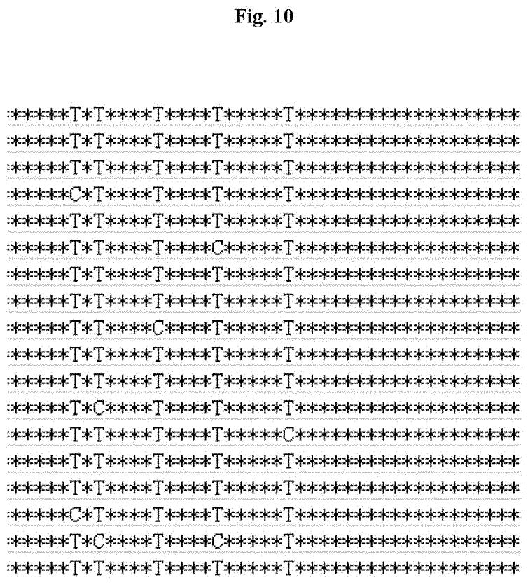

Methylation dimensionality detected by two different methods is compared, and the alignment result is as shown in a FIG. 9 and a FIG. 10.

FIG. 9 illustrates methylation level of each CpG locus detected by pyrophosphate detection method and FIG. 10 illustrates the methylation level (compared in a vertical direction of a same locus) of each CpG locus detected by the present disclosure and the methylation level (compared in a horizontal direction of a same sequence) on each DNA template molecule. It can be seen from FIG. 9 and FIG. 10 that methylation detection of the present disclosure can reflect more haplotype locus information compared with pyrosequencing.

It can be seen from the above description that the embodiments of the present disclosure achieve the following technical effects: the targeted region is amplified by using the improved primers of the present disclosure with high specificity and amplification efficiency, it can be convenient to construct the amplified targeted region to the amplicon library, and the methylation state is detected by the improved analytical flow, so that the number of the MGMT gene promoter methylation loci is improved, and therefore, the detection throughput and efficiency are improved and the detection accuracy is improved, thereby providing more reliable grounds for guiding medication.

A person skilled in the art should understand that the embodiment of the disclosure can be provided as a method, a system or a computer program product. Therefore, the present disclosure can adopt the forms of an entirely hardware embodiment, an entirely software embodiment or an embodiment combining hardware and software elements. Furthermore, the present disclosure can adopt the form of a computer program product which can be executed by one or more computer usable storage mediums containing computer usable program codes therein (including but not limited to magnetic disk memory, CD-ROM, optical memory and the like).

The present disclosure is described with reference to the flow diagrams and/or block diagrams of the method, equipment (system) and computer program product of the embodiments of the present disclosure. It should be understood that each flow and/or block in the flow diagram and/or the block diagram and combination of flow and/or block in the flow diagram and/or the block diagram can be implemented by computer program commands. These computer program commands can be provided to processors of a universal computer, a dedicated computer, an embedded processor or other programmable data processing devices to generate a machine, so that commands executed by the processors of the computer or other programmable data processing devices generate a device for implementing appointed functions in one or more flows in the flow diagram and/or one or more blocks in the block diagram.

The foregoing is merely illustrative of the preferred embodiments of the present disclosure and is not intended to be limiting of the present disclosure, and for those skilled in the art, the present disclosure may have various changes and modifications. Any modifications, equivalent substitutions, improvements, and the like within the spirit and principles of the disclosure are intended to be included within the scope of the present disclosure.

What is claimed is:

1. A next generation sequencing-based detection panel for glioma, the detection panel comprising glioma-related genes and loci, the glioma-related genes and loci comprising SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPM1D, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR vIII, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1;
preferably, the glioma-related genes and loci further comprise STR locus on chromosome 1 and STR locus on chromosome 19.

2. (canceled)

3. A next generation sequencing-based detection kit for glioma, comprising a detection probe and/or a detection primer directed at glioma-related genes and loci, the glioma-related genes and loci comprising SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPMID, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR viii, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1;
preferably, the glioma-related genes and loci further comprise STR locus on chromosome 1 and STR locus on chromosome 19;
preferably, the detection kit is configured to detect a plurality of mutation types, the plurality of mutation types comprising point mutation, fusion mutation, copy number variation, deletion mutation and insertion mutation;
preferably, the detection kit further comprising primers for detecting the MGMT promoter methylation, wherein the primers for detecting MGMT promoter methylation have sequences shown by SEQ ID NO: 1 and SEQ ID NO: 2;
preferably, the detection kit further comprising one or more of groups consisting of DNA library construction reagents, gene trapping reagents, bisulfite conversion reagents and gene amplification reagents;
preferably, the detection kit further comprising a glioma panel verification sample, the glioma panel verification sample comprising IDH1, IDH2, TERT, ABL1, ALK, BRAF, EGFR, FGFR2, FLT3, GNA11, GNA11, GNAQ, JAK2, KIT, KRAS, MEK1, MET, NOTCH, NRAS, PDGFRA, PIK3CA and NTRK gene standard substances.

4. (canceled)

5. (canceled)

6. (canceled)

7. (canceled)

8. (canceled)

9. The detection kit according to claim 3, further comprising a next generation sequencing-based system for detecting 1p/19q co-deletion of glioma, the next generation sequencing-based system for detecting 1p/19q co-deletion of glioma comprising an SNP locus selection device, an SNP detection device without a control sample and/or an SNP detection device with a control sample, wherein the SNP locus selection device is configured to select SNP loci on chromosome 1 and chromosome 19 according to public databases to obtain a first group of SNP loci; the SNP detection device without the control sample comprises:
a first sequencing module configured for sequencing a to-be-tested sample and a group of negative samples;
a first SNP detection module configured for detecting all SNP loci on chromosome 1 and chromosome 19 in the group of negative samples;
a first gSNP locus selection module configured for selecting gSNP loci of the group of negative samples in the first group of SNP loci;
a second SNP detection module configured for detecting all SNP loci on chromosome 1 and chromosome 19 in the to-be-tested sample;
a first calculation and statistics module configured for performing calculation and statistics on BAF of mutated gSNP loci on the gSNP loci determined in the first gSNP loci selection module in the to-be-tested sample and marking LOH status ratio (R¹) of the ith gSNP as |BAF−0.5| of the ith gSNP; and
a first judging module configured for correcting R on 1p and 19q of the to-be-tested sample according to R of the gSNP loci on 1q and 19p of the to-be-tested sample and determining a threshold value, judging an LOH status of each gSNP locus according to the threshold value and then judging co-deletion according to LOH statuses of all gSNP loci;
the SNP detection device with the control sample comprises:
a second sequencing module configured for sequencing the to-be-tested sample and a control sample;
a third SNP detection module configured for detecting all SNP loci on chromosome 1 and chromosome 19 in the control sample;
a second gSNP locus selection module configured for selection gSNP loci of the control sample in the first group of SNP loci;
a fourth SNP detection module configured for detecting all SNP loci on chromosome 1 and chromosome 19 in the to-be-tested sample;
a second calculation and statistics module configured for performing statistics on numbers of reads of reference sequence genotype and alternative sequence genotype of the control sample on the gSNP loci and marking as N₁ and N₂ respectively, performing statistics on numbers of reads of reference sequence genotype and alternative sequence genotype of the to-be-detected sample on the gSNP loci and marking as Tⱼ and T₂ respectively, calculating an LOH status ratio of each gSNP, wherein the LOH status (Rⁱ) of the ith gSNP is defined as follows:
$${R}^{i} = \vert \frac{\frac{{N}_{2}^{i}}{{N}_{1}^{i}}}{\frac{{N}_{2}^{i}}{{N}_{1}^{i}} + \frac{{T}_{2}^{i}}{{T}_{1}^{i}}} - 0.5 \vert ; and$$
a second judging module configured for correcting R on 1p and 19q of the to-be-tested sample according to R of the gSNP loci on 1q and 19p of the to-be-tested sample and determining a threshold value, judging an LOH status of each gSNP locus according to the threshold value and then judging co-deletion according to the LOH statuses of all gSNP loci;
preferably, the first gSNP locus selection module selects the gSNP loci of the group of negative samples in the first group of SNP loci according to the coverage, BAF and a fluctuation size of BAF in the group of negative samples, preferably, selection criteria of the gSNP loci are as follows: the coverage is greater than 100, the BAF range is 0.1-0.9 and max-min of BAF among samples in the group of negative samples is smaller than 0.2;
and preferably, the number of samples in the group of negative samples is greater than or equal to 30;
preferably, the second gSNP locus selection module selects the gSNP loci of the control sample in the first group of SNP loci according to the coverage and BAF, preferably, selection criteria of the gSNP loci are as follows: the coverage is greater than 100, and the BAF range is 0.3-0.7;
preferably, the public databases the public databases include: SNP138, 1000 Genomes Project and Chinese Millionome Database,
preferably, the SNP locus selection device select the SNP loci according to their allele frequency between 0.45-0.55 in the population;
and preferably, an SNP locus being selected at every 200 kb.

10. The detection kit according to claim 9, wherein the first judging module comprises:
a first statistics sub-module configured for performing statistics on mean values and variances of R in all gSNP loci in 1q and 19p respectively and calculating a Z value of each R on chromosome 1 and chromosome 19 respectively based on 1q and 19p;
a first threshold value calculation sub-module configured for calculating the Z values of the group of negative samples corrected by 1q and 19p and taking the mth percentile as a threshold value, preferably, m is greater than 95, and more preferably, m is equal to 99;
a first judging sub-module configured for comparing the Z value of each gSNP locus on 1p and 19q with the corresponding threshold value to judge an LOH status of the locus, judging the LOH status of the locus is abnormal if the Z value exceeds the threshold value, otherwise judging the LOH status of the locus is normal; and
a second judging sub-module configured for judging whether LOH occurs on 1p and 19q or not, performing statistics on numbers of abnormal and normal statuses on 1p and 19q respectively, judging that LOH of the sample occurs on 1p and 19q when abnormal statuses/(abnormal statuses+normal statuses) is greater than t₁, and judging the co-deletion of 1p and 19q of the sample occurs when only LOH occurs on 1p and 19q simultaneously, preferably, t₁ is greater than 0.6, and more preferably, t₁ is equal to 0.8.

11. (canceled)

12. The detection kit according to claim 9, wherein the second judging module comprises:
a second statistics sub-module configured for performing statistics on mean values and variances of R in all gSNP loci in 1q and 19p respectively and calculating a Z value of each R on chromosome 1 and chromosome 19 respectively based on 1q and 19p;
a second threshold value calculation sub-module configured for taking the mean values of Z values on 1q and 19p plus 2-6 times of variances respectively as threshold values of 1p and 19q;
a third judging sub-module configured for comparing the Z value of each gSNP locus on 1p and 19q with the corresponding threshold value to judge an LOH status of the locus, judging the LOH status of the locus is abnormal if the Z value exceeds the threshold value, otherwise judging the LOH status of the locus is normal;
a fourth judging sub-module configured for judging whether LOH occurs on 1p and 19q or not, performing statistics on numbers of abnormal and normal statuses on 1p and 19q respectively, judging that LOH of the sample occurs on 1p/19q when abnormal statuses/(abnormal statuses+normal statuses) is greater than t₂, and judging co-deletion of 1p and 19q of the sample occurs when only LOH occurs on 1p and 19q simultaneously, preferably, t₂ is greater than 0.6, and more preferably, t₂ is equal to 0.9.

13. (canceled)

14. (canceled)

15. The detection kit according to claim 9, wherein the system comprises a first verification device, the first verification device being configured to detect co-deletion of 1p and 19q based on STR and the first verification device comprising:
an STR acquisition module configured for extracting known STR from existing data;
a control sample STR statistics module configured for extracting reads covering the known STR from an alignment result file of the control sample, performing statistics on the number of repeat unit of the known STR on each read, extracting the two repeat units with the most numbers for each STR, marking as N₃ and N₄, wherein if N₃/N₄ is greater than n, it is considered that the STR is a homozygotic type and is no longer configured for result judgment; and preferably, n is greater than 5, and more preferably, n is equal to 10;
a to-be-tested sample STR statistics module configured for extracting reads covering the known STR from an alignment result file of the to-be-tested sample, performing statistics the number of reads on the two repeat units determined in the control sample STR statistics module marking as T₃ and T₄ and calculating an LOH status of each STR, wherein the LOH status (R) of the ith STR is defined as follows:
$${R}^{i} = \frac{{T}_{4}^{i} / {T}_{3}^{i}}{{N}_{4}^{i} / {N}_{3}^{i}} ;$$
and
a third judging module configured for correcting R on 1p and 19q of the to-be-tested sample and determining a threshold value, judging an LOH status of each STR locus according to the threshold value and then judging co-deletion according to LOH statuses of all STR loci;
preferably, the reads covering the known STR are reads covering from the upstream 20 bp to the downstream 20 bp of the known STR;
preferably, the system comprises a second verification device, the second verification device being configured to detect co-deletion of 1p and 19q based on CNV.

16. (canceled)

17. The detection kit according to claim 15, wherein the third judging module comprises:
a fifth judging sub-module configured for judging LOH status of each STR and judging that LOH status of the locus is abnormal when R is smaller than T, otherwise, judging that LOH status of the locus is normal, preferably, T is equal to 0.5; if R is greater than 1, converting to 1/R;
a sixth judging sub-module configured for judging whether LOH occurs on 1p and 19q or not, performing statistics on numbers of abnormal and normal statuses on 1p and 19q respectively, judging that LOH of the sample occurs on 1p/19q when abnormal statuses/(abnormal statuses+normal statuses) is greater than t₃, and judging co-deletion of 1p and 19q of the sample when only LOH occurs on 1p and 19q simultaneously, preferably, t₃ is greater than 0.6, and more preferably, t₃ is equal to 0.8.

18. (canceled)

19. The detection kit according to claim 3, further comprising a processing device for sequencing data of MGMT gene promoter methylation, wherein the processing device for sequencing data of MGMT gene promoter methylation comprises:
an acquisition module configured for acquiring methylated sequencing data originated from an MGMT gene promoter, wherein the methylated sequencing data is a double-end sequencing sequence;
an alignment module configured for aligning the methylated sequencing data with a human reference genomic sequence to obtain an alignment result, the alignment result comprising a first end first matching region, a first end second matching region, a second end first matching region and a second end matching region, wherein the first end second matching region and the second end second matching region are overlapped;
a removal module configured for removing the first end second matching region or the second end second matching region in the alignment result to obtain to-be-analyzed data; and
a methylation recognition module configured for methylated locus recognition in the to-be-analyzed data to obtain a methylated result of the MGMT gene promoter;
preferably, the processing device further comprises:
a first pre-treatment module configured for pre-treatment of converting the human reference genomic sequence from C to T; and
a second pre-treatment module configured for pre-treatment of converting the double-end sequencing sequence from C to T;
preferably, the processing device further comprises a correction module configured for correcting the to-be-analyzed data, the correction module being configured to correct the to-be-analyzed data by means of the human reference genomic sequence, position information of the human reference genomic sequence and high frequency SNP loci in a population.

20. (canceled)

21. (canceled)

22. The detection kit according to claim 19, wherein the methylated recognition module comprises:
a primary identification module configured for identifying the methylated loci in the to-be-analyzed data primarily to obtain primarily identified loci; and
a confidence selection module configured for confidence selection of the primarily identified loci to obtain a methylated result of the MGMT gene promoter, wherein criteria for confidence selection are as follows: a coverage is smaller than 3000000, a possibility ratio standard between optimum and sub-optimum genotypes is greater than or equal to 20 and a comparison mass is greater than 5.

23. (canceled)

24. (canceled)

25. A method for detecting glioma, the method comprising detecting glioma-related genes and loci by using a detection probe and/or a detection primer, the glioma-related genes and loci comprising SNP locus on chromosome 1, SNP locus on chromosome 19, MGMT, ATRX, H3F3A, ACVR1, CTC, HIST1H3B, MLH1, PLCG1, SMO, AKT1, CTNNB1, HIST1H3C, MSH2, PMS2, TERT, ATRX, DAXX, HRAS, MSH6, PPM1D, TP53, BCOR, DDX3X, IDH1, MYC, PTCH1, TRAF7, BRAF, EGFR, IDH2, MYCN, PTEN, TSC1, BRCA1, FAT1, KDR, NF1, PTPN11, TSC2, BRCA2, FGFR1, KIT, NF2, RB1, USP8, CDK4, FGFR3, KLF4, NOTCH1, RELA, YAP1, CDK6, FUBP1, KRAS, NRAS, RGPD3, CDKN2A, GNAQ, MDM4, PDGFRA, SETD2, CDKN2B, GNAS, MEN1, PIK3CA, SMARCB1, CHEK2, H3F3A, MET, PIK3R1, SMARCE1, EGFR viii, NTRK3, TYMS, NTRK1, NTRK2, GSTP1, ABCB1, CYP2B6, CYP2C19, DHFR, DYNC2H1, ERCC1, MTHFR, SLIT1, SOD2, UGT1A1 and XRCC1;
preferably, the glioma-related genes and loci further comprise STR locus on chromosome 1 and STR locus on chromosome 19;
preferably, the method further comprising detecting a plurality of mutation types, the plurality of mutation types comprising point mutation, fusion mutation, copy number variation, deletion mutation and insertion mutation;
preferably, the method further comprising detecting MGMT promoter methylation, wherein primers for detecting the MGMT promoter methylation have sequences shown by SEQ ID NO: 1 and SEQ ID NO: 2.

26. (canceled)

27. (canceled)

28. (canceled)

29. The method according to claim 25, further comprising detecting 1p/19q co-deletion of glioma based on next generation sequencing, the detecting 1p/19q co-deletion of glioma based on next generation sequencing comprising selecting SNP loci and performing SNP detection without a control sample and/or SNP detection with a control sample, wherein the selecting the SNP loci comprises selecting the SNP loci on chromosome 1 and chromosome 19 of human according to the public databases to obtain a first group of SNP loci; and the SNP detection without the control sample comprises:
S11, sequencing a to-be-tested sample and a group of negative samples;
S12, detecting all SNP loci on chromosome 1 and chromosome 19 in the group of negative samples;
S13, selecting gSNP loci of the group of negative samples in the first group of SNP loci;
S14, detecting all SNP loci on chromosome 1 and chromosome 19 in the to-be-tested sample;
S15, performing calculation and statistics on BAF of mutated gSNP loci on the gSNP loci determined in the S13 in the to-be-tested sample and marking an LOH status ratio (R¹) of the ith gSNP as |BAF−0.5| of the ith gSNP; and
S16, correcting R on 1q and 19p of the to-be-tested sample according to R of the gSNP loci on 1p and 19q of the to-be-tested sample and determining a threshold value, judging an LOH status of each gSNP locus according to the threshold value and then judging co-deletion according to LOH status of all gSNP loci;
the SNP detection with the control sample comprises:
S21, sequencing the to-be-tested sample and the control sample;
S22, detecting all SNP loci on chromosome 1 and chromosome 19 in the control sample;
S23, selecting gSNP loci of the control sample in the first group of SNP loci;
S24, detecting all SNP loci on chromosome 1 and chromosome 19 in the to-be-tested sample;
S25, performing statistics on numbers of reads of reference sequence genotype and alternative sequence genotype of the control sample on the gSNP loci and marking as N₁ and N₂ respectively, performing statistics on numbers of reads of reference sequence genotype and alternative sequence genotype of the to-be-detected sample on the gSNP loci and marking as T₁ and T₂ respectively, calculating an LOH status ratio of each gSNP, wherein an LOH status (Rⁱ) of the ith gSNP is defined as follows:
$${R}^{i} = \vert \frac{\frac{{N}_{2}^{i}}{{N}_{1}^{i}}}{\frac{{N}_{2}^{i}}{{N}_{1}^{i}} + \frac{{T}_{2}^{i}}{{T}_{1}^{i}}} - 0.5 \vert ;$$
and
S26, correcting R on 1q and 19p of the to-be-tested sample according to R of the gSNP loci on 1p and 19q of the to-be-tested sample and determining a threshold value, judging an LOH status of each gSNP locus according to the threshold value and then judging co-deletion according to LOH statuses of all gSNP loci.

30. The method according to claim 29, wherein the S16 comprises:
S161, performing statistics on mean values and variances of R in all gSNP loci in 1q and 19p respectively and calculating a Z value of each R on chromosome 1 and chromosome 19 respectively based on 1q and 19p;
S162, calculating Z values of the group of negative samples corrected by 1q and 19p and taking the mth percentile as a threshold value, preferably, m is greater than 95, and more preferably, m is equal to 99;
S163, comparing the Z value of each gSNP locus on 1p and 19q with the corresponding threshold value to judge an LOH status of the locus, judging the LOH status of the locus is abnormal if the Z value exceeds the threshold value, otherwise judging the LOH status of the locus is normal;
S164, judging whether LOH occurs on 1p and 19q or not, performing statistics on numbers of abnormal and normal statuses on 1p and 19q respectively, judging that LOH of the sample occurs on 1p and 19q when abnormal statuses/(abnormal statuses+normal statuses) is greater than t₁, and judging co-deletion of 1p and 19q of the sample when only LOH occurs on 1p and 19q simultaneously, preferably, t₁ is greater than 0.6, and more preferably, t₁ is equal to 0.8.

31. The method according to claim 29, wherein the S13 comprises selecting the gSNP loci of the group of negative samples in the first group of SNP loci according to a coverage, BAF and a fluctuation size of BAF in the group of negative samples, preferably, selection criteria of the gSNP loci is as follows: the coverage is greater than 100, the BAF range is 0.1-0.9 and max-min of BAF among samples in the group of negative samples is smaller than 0.2;
and preferably, the number of samples in the group of negative samples is greater than or equal to 30.

32. The method according to claim 29, wherein the S26 comprises:
S261, performing statistics on mean values and variances of R in all gSNP loci in 1q and 19p respectively and calculating a Z value of each R on chromosome 1 and chromosome 19 respectively based on 1q and 19p;
S262, taking the mean values of Z values on 1q and 19p plus 2-6 times of variances respectively as threshold values of 1p and 19q;
S263, comparing the Z value of each gSNP locus on 1p and 19q with the corresponding threshold value to judge an LOH status of the locus, judging the LOH status of the locus is abnormal if the Z value exceeds the threshold value, otherwise judging the LOH status of the locus is normal;
and S264, judging whether LOH occurs on 1p and 19q or not, performing statistics on numbers of abnormal and normal statuses on 1p and 19q respectively, judging that LOH of the sample occurs on 1p/19q when abnormal statuses/(abnormal statuses+normal statuses) is greater than t₂, and judging co-deletion of 1p and 19q of the sample when only LOH occurs on 1p and 19q simultaneously, preferably, t₂ is greater than 0.6, and more preferably, t₂ is equal to 0.9.

33. The method according to claim 29, wherein the S23 comprises selecting the gSNP loci of the control sample in the first group of SNP loci according to a coverage and BAF, preferably, selection criteria of the gSNP loci is as follows: the coverage is greater than 100, the BAF range is 0.3-0.7.

34. The method according to claim 29, wherein the public databases comprise SNP138, 1000 Genomes Project and Chinese Millionome Database,
preferably, the selecting SNP locus comprises selecting the loci SNP loci according to their allele frequency between 0.45-0.55 in the population;
and preferably, an SNP locus being selected at every 200 kb.

35. The method according to claim 29, further comprising a first verification step, the first verification step being configured to detect co-deletion of 1p and 19q based on STR and the first verification step comprising:
S31, extracting known STR from existing data;
S32, extracting reads covering the known STR from an alignment result file of the control sample, performing statistics on the number of repeat unit of the known STR on each read, extracting the two repeat units with the most numbers for each STR, marking as N₃ and N₄, wherein if
$$\frac{{N}_{3}}{{N}_{4}}$$
is greater than n, it is considered that the STR is a homozygotic type and is no longer configured for result judgment; and preferably, n is greater than 5, and more preferably, n is equal to 10;
S33, extracting reads covering the known STR from an alignment result file of the to-be-tested sample, performing statistics the number of reads on the two repeat units determined in the control sample STR statistics module marking as T₃ and T₄ and calculating an LOH status of each STR, wherein the LOH status (Rⁱ) of the ith STR is defined as follows:
$${R}^{i} = \frac{{T}_{4}^{i} / {T}_{3}^{i}}{{N}_{4}^{i} / {N}_{3}^{i}} ;$$
and
S34, correcting R on 1p and 19q of the to-be-tested sample and determining a threshold value, judging an LOH status of each STR locus according to the threshold value and then judging co-deletion according to LOH statuses of all STR loci;
preferably, the reads covering the known STR are reads covering from the upstream 20 bp to the downstream 20 bp of the known STR;
preferably, the S34 comprises:
S341, judging the LOH status of each STR and judging that the LOH status of the locus is abnormal when R is smaller than T, otherwise, judging that the LOH status of the locus is normal, preferably, T is equal to 0.5; if R is greater than 1, converting to 1/R;
and S342, judging whether LOH occurs on 1p and 19q or not, performing statistics on numbers of abnormal and normal statuses on 1p and 19q respectively, judging that LOH-1 of the sample occurs on 1p/19q when abnormal statuses/(abnormal statuses+normal statuses) is greater than t₃, and judging co-deletion of 1p and 19q of the sample when only LOH occurs on 1p and 19q simultaneously, preferably, t₃ is greater than 0.6, and more preferably, t₃ is equal to preferably, the method further comprising a second verification step, the second verification step being configured to detect co-deletion of 1p and 19q based on CNV.

36. (canceled)

37. (canceled)

38. (canceled)

39. The method according to claim 29, further comprising the sequencing data of MGMT gene promoter methylation, wherein sequencing data of MGMT gene promoter methylation comprises:
acquiring methylated sequencing data originated from an MGMT gene promoter, wherein the methylated sequencing data is a double-end sequencing sequence;
aligning the methylated sequencing data with a human reference genomic sequence to obtain an alignment result, the alignment result comprising a first end first matching region, a first end second matching region, a second end first matching region and a second end matching region, wherein the first end second matching region and the second end second matching region are overlapped;
removing the first end second matching region or the second end second matching region in the alignment result to obtain to-be-analyzed data; and
performing methylated locus recognition in the to-be-analyzed data to obtain a methylated result of the MGMT gene promoter;
preferably, before aligning the methylated sequencing data with the human reference genomic sequence, the sequencing data of MGMT gene promoter methylation further comprises:
pre-treatment of converting the human reference genomic sequence from C to T; and
pre-treatment of converting the double-end sequencing sequence from C to T.

40. (canceled)

41. The method according to claim 39, wherein after obtaining the to-be-analyzed data and before performing methylated loci recognition on the to-be-analyzed data, the sequencing data of MGMT gene promoter methylation further comprises a step of correcting the to-be-analyzed data, the step of correcting the to-be-analyzed data comprising:
correcting the to-be-analyzed data by means of the human reference genomic sequence, position information of the human reference genomic sequence and high frequency SNP loci of a crowd.

42. The method according to claim 39, wherein the step of performing methylated locus recognition in the to-be-analyzed data to obtain a methylated result of the MGMT gene promoter comprises:
identifying the methylated loci in the to-be-analyzed data primarily to obtain primarily identified loci; and
confidence selection of the primarily identified loci to obtain a methylated result of the MGMT gene promoter,
wherein criteria for the confidence selection are as follows: a coverage is smaller than 3000000, a possibility ratio standard between optimum and sub-optimum genotypes is greater than or equal to 20 and a comparison mass is greater than 5.
